(19) United States
(12) Patent Application Publication
(10) Pub. No.: US 2019/0001163 A1
(43) Pub. Date: Jan. 3, 2019

(54) COMPOSITIONS CONTAINING POLYCARBODIIMIDE COMPOUNDS AND ACIDS

(71) Applicant: L'OREAL, Paris (FR)

(72) Inventors: Ronak RUGHANI, Edison, NJ (US); ChoCho KHINE, Bridgewater, NJ (US); Andrea ELSEN-WAHRER, Linden, NJ (US)

(73) Assignee: L'OREAL, Paris (FR)

(21) Appl. No.: 15/636,891

(22) Filed: Jun. 29, 2017

(51) Int. Cl. A61Q 5/06; A61Q 5/00; A61K 8/40; A61K 8/84; A61K 8/891

(52) U.S. Cl. CPC A61Q 5/065; A61Q 5/002; A61K 2800/4322; A61K 8/84; A61K 8/891; A61K 8/40

(57) ABSTRACT

Disclosed are compositions and agents for treating keratinous fibers including a polycarbodiimide compound in various combinations with a carboxylic acid, an amine and a carboxysilicone polymer in order to improve the quality of the keratinous fibers such as hair. In particular, agents, kits, and methods for imparting protection to hair and/or minimizing hair damage caused by extrinsic and intrinsic factors and for improving the condition of or repairing damaged hair are disclosed.

### FIELD OF THE INVENTION

The present invention relates to compositions, agents, and methods for treating keratinous fibers. More particularly, the present invention relates to keratinous fiber treatment compositions that can repair or prevent/minimize damaging effects on keratinous fibers caused by extrinsic and intrinsic factors by providing desirable or improved quality and conditioning, hydrophobicity, and improved mechanical properties of keratinous fibers, in particular, hair.

### BACKGROUND OF THE INVENTION

The appearance and/or condition of keratinous substrates, for example, hair, skin, nails, and lips, are often affected by both extrinsic and intrinsic factors such as aging. In particular, when keratinous substrates are exposed to environmental conditions, for example, high or low humidity or to ultraviolet radiation from the sun, these substrates can lose many of their desirable properties and even become damaged. Keratinous substrates comprising keratinous fibers, especially hair, are constantly exposed to harsh extrinsic conditions, such as sun, chemical damage, e.g., from detergents in shampoos, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or hair breakage and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. In addition, hair fibers can become less strong, thereby breaking easily under stress such as stress induced by pulling, brushing, or combing activities. Furthermore, hair can lose its desirable attributes due to physical stress on the hair such as brushing and application of heat. The magnitude of the consequences of these factors is variable, depending on, for example, the quality of the hair, length, style, and environmental factors. As such, these factors generally result in damage to the keratinous fibers, either by affecting protective materials on the surface of the hair (the cuticle), or by altering the hair fiber internally (the cortex).

More specifically, extrinsic conditions may strip protective materials from the surface of the hair, and/or they may disrupt the organized structure of the hair fibers, called the α-structure, which may be accompanied by a decrease in the tensile strength. Such damage to hair by extrinsic factors is more evident the further the hair fiber has grown from the root, because the hair has been exposed longer to such extrinsic factors. In effect, the hair has what may be called a “damage history” as it grows, i.e., the further from the root, the lower the tensile strength and the greater the breakdown in α-structure that has occurred. As a result, consumers continue to seek products such as hair care and hair cosmetic compositions which protect and enhance the appearance of hair as well as reduce the deleterious effects of adverse environmental conditions, photo-damage, and physical stress. Consumers also desire to use hair chemical treatments such as hair dyes, hair relaxers, perm and wave treatments, hair bleaches/lighteners and highlighting treatments that are less damaging to the hair.

Morphologically, a hair fiber contains four structural units: cuticle, cortex, medulla, and intercellular cement. The cuticle layers are located on the hair surface and consist of flat overlapping cells (“scales”). These scales are attached at the root end and point toward the distal (tip) end of the fiber and form layers around the hair cortex. The cortex comprises the major part of the hair fiber. The cortex consists of spindle-shaped cells, or macrofibrils, that are aligned along the fiber axis. The macrofibrils further consist of microfibrils (highly organized protein units) that are embedded in the matrix of amorphous protein structure. The medulla is a porous region in the center of the fiber. The medulla is a common part of wool fibers but is found only in thicker human hair fibers. Finally, the intercellular cement is the material that binds the cells together, forming the major pathway for diffusion into the fibers.

The mechanical properties of hair are determined by the cortex. A two-phase model for the cortex organization has been suggested. Milczarek et al, Colloid Polym. Sci., 270, 1106-1115 (1992). In this model, water-impenetrable microfilaments (“rods”) are oriented parallel with the fiber axis. The microfilaments are embedded in a water-penetrable matrix (“cement”). Within the microfilaments, coiled protein molecules are arranged in a specific and highly organized way, representing a degree of crystallinity in the hair fiber.

Similar to other crystalline structures, hair fibers display a distinct diffraction pattern when examined by wide-angle X-ray diffraction. In normal, non-stretched hair fibers this pattern is called an “alpha-pattern”. The alpha-pattern or α-structure of hair is characterized by specific repeated spacings (9.8 Å, 5.1 Å, and 1.5 Å), All proteins that display this X-ray diffraction pattern are called α-proteins and include, among others, human hair and nails, wool, and porcupine quill. When the hair fiber is stretched in water, a new X-ray diffraction pattern emerges that is called a “β-pattern”, with new spacings (9.8 Å, 4.65 Å, and 3.3 Å).

Damage to hair may occur in the cuticle and/or the cortex. When normal hair is damaged by heat, chemical treatment, UV radiation, and/or physical/mechanical means, myriad chemical and physical changes are induced in the hair. For example, these damaging processes have been known to produce removal or damage to cuticle scales or to cleave the thioester linkage holding the hydrophobic 18-methyl eicosanoic acid (“18-MEA”) layer to hair. Thus, it is commonly observed that undamaged hair exhibits significant hydrophobic character, whereas damaged hair shows significant hydrophilic character due to the removal of surface lipids.

There is a need, therefore, for cosmetic products that are useful in protecting the chemical and physical structure of keratinous fibers from harsh extrinsic conditions and restoring the hair's physical properties to undamaged states following damage by extrinsic conditions. More particularly, there is a need to find materials or compositions or methods that can provide a protective barrier and/or treatment to hair to protect it at the cortex. Such a protective barrier or treatment should not be easily transferred from the substrate over time by normal everyday activity. Non-transfer, wash or water-resistant cosmetic, hair and skin care compositions are sought which have the advantage of forming a deposit which does not undergo even partial transfer to the substrates with which they are brought into contact (for example, clothing). It is also desirable to have compositions that do not easily “run off” or wash off the skin and lips when exposed to water, rain or tears. Accordingly, a product that imparts a protective barrier to the substrate that also is shampoo, wash or water/humidity resistant and non-transferable would be of benefit to the area of cosmetic products. As such, makers of cosmetic products such as hair and skin care products continue to seek materials and ingredients that can provide such benefits. At the same time, long lasting benefits or durability of these benefits are also desirable.

In addition, in today's market, many consumers prefer the flexibility of having products that can be used on hair or skin on different ways. Methods for caring for or non-permanent shaping of keratinous fibers include, for example, brushing, teasing, braiding, the use of hair rollers, and heat styling, optionally with a commercially available hair care and styling products. Non-limiting examples of heat styling include blow drying, crimping, curling, and straightening methods using elevated temperatures (such as, for example, setting hair in curlers and heating, and curling with a curling iron and/or hot/steam rollers and/or flat iron).

There is a need, therefore, for materials, compositions, treatment systems, and methods that result in hair with improved quality or that is less damaged when the hair is exposed to adverse environmental and physical factors and/or when chemically treated. As such, it is also advantageous to find a means for treating damaged keratinous fibers by repairing them, that is to say by intrinsically improving the condition of the keratinous fibers, reducing and/or preventing breakage of the keratinous fibers. At the same time, it is desirable that said materials, compositions, treatment systems, and methods provide durable or long-lasting caring and repair benefits to hair.

To achieve at least one of these and other advantages, the present invention provides methods of protecting and/or repairing a keratinous substrate, and more particularly, a keratinous fiber chosen from hair, eyelashes and eyebrows from extrinsic damage caused by heating, UV radiation, chemical treatment or other harsh treatment by applying to or contacting said keratinous fiber according to the methods of the invention, with compositions that include a polycarbodiimide compound, a carboxylic acid, and in some embodiments, an amine, and/or a carboxysilicone polymer in various combinations thereof in amounts effective to repair or reduce/prevent damage to or to prevent/minimize breakage of or improve the quality and condition of the keratinous fiber as well as to impart hydrophobicity or increased hydrophobicity to the fiber.

### BRIEF SUMMARY OF THE INVENTION

In an exemplary embodiment, the present disclosure relates to a keratinous fiber treatment agent comprising one or more separately contained treatment compositions, wherein each of the one or more treatment compositions include at least one of the following components:
(a) at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and mixtures thereof;
(b) at least one amine chosen from alkyl amines, alkanolamines, alkylene amines, and mixtures thereof;
(c) at least one polycarbodiimide compound; and
(d) at least one carboxysilicone polymer;
   provided that components (a) and (c) are included in the one or more treatment compositions.

The agents for treating keratinous fibers include various treatment compositions containing amounts of each of the at least one carboxylic acid, the at least one amine, the at least one polycarbodiimide compound, and the at least one carboxysilicone polymer sufficient to repair or reduce/prevent damage to or to prevent/minimize breakage of or improve the quality and condition of keratinous fibers such as hair, as well as to impart hydrophobicity or increased hydrophobicity to hair that has been damaged or adversely affected by extrinsic and/or intrinsic factors. These factors are, for example, before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents) or when subjecting the hair to heat and other stresses (brushing, combing, or shampooing with detergents).

In exemplary embodiments, the present invention is directed to treatment agents, compositions, and methods for protecting keratinous fibers from or repairing said fiber or to prevent/minimize breakage of or improve the quality and condition of keratinous fibers such as hair as well as to impart hydrophobicity or increased hydrophobicity to the hair following extrinsic damage caused by heating, UV radiation or chemical treatment. The methods include applying to the keratinous fibers according to one-step or multi-step application methods, various treatment compositions including at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, at least one amine chosen from alkyl amines, alkanolamines, alkylene amines, and mixtures thereof, at least one polycarbodiimide compound, and at least one carboxysilicone polymer.

In an embodiment, the methods of the present disclosure involve a one-step treatment of keratinous fibers comprising applying a first treatment composition containing at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, and at least one polycarbodiimide compound.

In some exemplary embodiments, the methods of the present disclosure involve a one-step treatment of keratinous fibers comprising applying a first treatment composition containing at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, at least one amine chosen from alkyl amines, alkanolamines, alkylene amines, and mixtures thereof, and at least one polycarbodiimide compound.

In some exemplary embodiments, the methods of the present disclosure involve a step-wise application treatment (two-step application) of keratinous fibers including: (1) applying a first treatment composition containing at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, at least one amine chosen from alkyl amines, alkanolamines, alkylene amines, and mixtures thereof, and at least one polycarbodiimide compound; (2) heating the treated fibers; and (3) applying a second treatment composition containing at least one polycarbodiimide compound and at least one carboxysilicone polymer.

In some exemplary embodiments, the methods of the present disclosure involve a step-wise application treatment (three-step application) of keratinous fibers including: (1) applying a first treatment composition containing at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, and at least one amine chosen from alkyl amines, alkanolamines, alkylene amines, and mixtures thereof; (2) applying a second treatment composition containing at least one polycarbodiimide compound; (3) applying a third treatment composition containing at least one carboxysilicone polymer; and (4) heating the fibers.

In some exemplary embodiments, the methods of the present disclosure involve a step-wise application treatment of keratinous fibers including: (1) applying a first treatment composition containing at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, at least one amine chosen from alkyl amines, alkanolamines, alkylene amines, and mixtures thereof; and at least one hair active agent; (2) rinsing the fibers; and (3) applying a second treatment composition containing at least one polycarbodiimide compound and at least one carboxysilicone polymer. The hair active agents may be chosen from bleaching agents, oxidizing agents, coloring agents, relaxing agents, straightening agents, perming agents, waving agents, and mixtures thereof. Such methods may include a step of applying a conditioning composition containing at least one conditioning agent chosen from cationic conditioning agents, silicone compounds, and mixtures thereof.

According to some embodiments, the present disclosure relates to kits which include separate packaging of the one or more treatment compositions provided in one or more of thickened or un-thickened aqueous and non-aqueous phases, and packaging of any of the foregoing with one or more additional ingredients that are known and commonly used in cosmetic compositions

In particular, the keratinous fiber such as hair that is treated via the compositions, methods and systems according to the invention can result in stronger and less brittle hair and/or less hair breakage and/or increased hydrophobicity on the surface of the hair fibers as well as result in hair that remains well-behaved or is more manageable (less frizzy or no frizziness). Thus, hair fibers are aligned, smooth and disentangle easily, which makes them easier to comb. The treated hair can also have more body (it is not limp) and is thus easier to style or shape.

The compositions, methods, and systems for treating hair according to the invention have the advantage of imparting durable or long-lasting benefits to the hair.

Other features and advantages of the present invention will be apparent from the following more detailed description of the exemplary embodiment which illustrates, by way of example, the principles of the invention.

### BRIEF DESCRIPTION OF THE DRAWINGS

Implementation of the present technology will now be described, by way of example only, with reference to the attached figures, wherein:

FIG. 1 shows plateau stress measurements obtained from tensile testing conducted on hair fibers treated with test compositions according to a one-step application method involving maleic acid-monoethanolamine-polycarbodiimide containing compositions.

FIG. 2 shows break stress measurements obtained from tensile testing conducted on hair fibers treated with test compositions according to a one-step application method involving maleic acid-monoethanolamine-polycarbodiimide containing compositions.

FIG. 3 shows toughness measurements obtained from tensile testing conducted on hair fibers treated with test compositions according to a one-step application method involving maleic acid-monoethanolamine-polycarbodiimide containing compositions.

FIG. 4 shows cycles to break measurements obtained from tensile testing conducted on hair fibers treated with test compositions according to a two-step application method involving maleic acid-monoethanolamine-polycarbodiimide and polycarbodiimide-carboxysilicone containing compositions.

FIG. 5 shows plateau stress measurements obtained from tensile testing conducted on hair fibers treated with test compositions according to a three-step application method involving maleic acid-monoethanolamine, polycarbodiimide and carboxysilicone containing compositions.

FIG. 6 shows break stress measurements obtained from tensile testing conducted on hair fibers treated with test compositions according to a three-step application method involving maleic acid-monoethanolamine, polycarbodiimide and carboxysilicone containing compositions.

FIG. 7 shows toughness measurements obtained from tensile testing conducted on hair fibers treated with test compositions according to a three-step application method involving maleic acid-monoethanolamine, polycarbodiimide and carboxysilicone containing compositions.

FIG. 8 shows cycles to break measurements obtained from tensile testing conducted on hair fibers treated with test compositions according to a three-step application method involving maleic acid-monoethanolamine, polycarbodiimide and carboxysilicone containing compositions.

It should be understood that the various aspects are not limited to the arrangements and instrumentality shown in the drawings.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

The term “comprising” (and its grammatical variations) as used herein is used in the inclusive sense of “having” or “including” and not in the exclusive sense of “consisting only of.”

“Conditioning,” as used herein, means imparting at least one of combability, manageability, moisture-retentivity, luster, shine, softness, and body to the hair.

“Durable conditioning,” as used herein, means that, following at least one shampoo/washing/rinsing after treatment of keratinous fibers such as hair with the compositions of the present disclosure, treated hair still remains in a more conditioned state as compared to untreated hair. The state of conditioning can be evaluated by measuring and comparing, the ease of combability of the treated hair and of the untreated hair in terms of ease of combing and/or the substantivity of the conditioning agent on the hair and/or the hydrophobicity of hair which can be assessed by contact angle measurements (spread of a water droplet on the surface of the hair).

“Heating” refers to the use of elevated temperature (i.e., above room temperature such at or above 30 C). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hair dryers, hood dryers, heating caps and steamers.

“High humidity,” as defined herein, refers to atmospheric humidity above 40%.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “style” or styling” as used herein includes shaping, straightening, curling, or placing a keratin fiber such as hair, in a particular arrangement, form or configuration; or altering the curvature of a keratinous fiber or other substrate; or re-positioning a keratin fiber or other substrate to a different arrangement, form or configuration; or providing/maintaining a hold to the shape or configuration of the keratin fiber. In some embodiments, the hold to the shape of configuration of the fiber may be expressed as an improved bending force property.

The term “treat” (and its grammatical variations) as used herein refers to the application of the compositions of the present invention onto keratinous substrates such as keratinous fibers or hair or skin or to contacting said keratinous substrates with the compositions of the present invention.

The term “repair” (and its grammatical variations) as used herein means that the damaged keratinous fibers such as hair fibers following treatment with the compositions of the present disclosure showed an improvement in tensile properties which are similar to that of natural undamaged hair. The improvement in tensile properties can be determined or assessed by any available means such as by mechanical tests of the fibers or by consumer and sensory evaluations of perceivable fiber strengthening attributes which have a positive impact on fiber mechanical properties. The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It is an object of the present invention to provide materials and compositions and methods which provide both a protective barrier or repairing treatment onto keratinous substrates such as hair and which impart native/undamaged physical properties-such as cortex repair, strength, less breakage, hydrophobicity, ease of combing, conditioning, anti-frizz, etc.—to hair, in particular, damaged hair, as well as impart durable or long lasting physical properties mimicking natural/undamaged hair to damaged hair.

It is also an object of the present invention to provide materials and compositions and methods which protect or repair a keratinous fiber chosen from hair comprising applying to the keratinous fiber the composition of the present invention in an amount effective to protect or repair said keratinous fiber before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

As such, it is desirable to formulate hair repair strategies to address the need for both surface and cortex repair. The present invention directed to compositions, methods and agents employing a carbodiimide polymer, a carboxylic acid, an amine and a carboxysilicone in various combinations thereof was found to provide a durable or long lasting coating on the surface of damaged keratinous substrates such as hair fiber, as well as mechanical strength to the fiber that yields a repaired/reinforce fiber that is able to withstand day-to-day grooming and hydrophobicity/conditioning/combability properties to the fibers.

In an embodiment, the present disclosure is directed to an agent for treating keratinous fibers comprising one or more separately contained treatment compositions, wherein each of the one or more treatment compositions include at least one of the following components:
(a) at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and mixtures thereof;
(b) at least one amine chosen from alkyl amines, alkanolamines, alkylene amines, and mixtures thereof;
(c) at least one polycarbodiimide compound; and
(d) at least one carboxysilicone polymer;
   provided that components (a) and (c) are included in the one or more treatment compositions.

In an embodiment, the at least one amine is chosen from monoethanolamine (MEA), diethanolamine (DEA), triethanolamine (TEA), monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, 3-isopropoxypropylamine, 3-methoxypropylamine (3-MPA), tris(hydroxymethyl)aminomethane, 3-ethoxypropylamine, 3-(2-ethylhexyloxy)-propylamine, 2-(2-aminoethoxy)ethanol (2-2AEE), 3-butoxypropylamine (3-BPA), ethylamines, ethyleneamine, and mixtures thereof, and is preferably chosen from monoethanolamine.

In an embodiment, the at least one polycarbodiimide compound has the following formula:

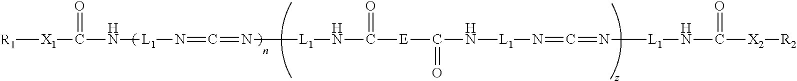

wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:

O—R₃—O; S—R₄—S; and

R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

In an embodiment, the at least one carboxysilicone polymer is a compound having at least one carboxylate group and is chosen from having the formula:

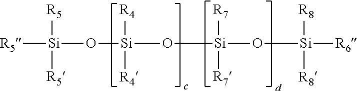

wherein the radicals R₄ are identical to or different from each other and are chosen from a linear or branched C₁-C₂₂ alkyl radical, a C₁-C₂₂ alkoxy radical and a phenyl radical, the radicals R₅, R₅′, R₅″, R₆, R₆′, R₆″, R₇, and R₇′ are identical to or different from each other and are chosen from a linear or branched C₁-C₂₂ alkyl radical, a C₁-C₂₂ alkoxy radical, a phenyl radical, a radical —(R₁O)ₐ—R₂—(OR₃)_{b}—COOM, a radical containing pyrrolidone carboxylic acid, a radical of polyvinyl acid/ester; and
wherein at least one of the radicals R₅, R₆ and R₇ is a radical chosen from a radical —(R₁O)ₐ—R₂—(OR₃)_{b}—COOM, a radical containing pyrrolidone carboxylic acid, a radical of polyvinyl acid/ester, a radical of polyvinyl acid/ester;
wherein R1 and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene;
   wherein c and d are integers from 0 to 1000, the sum c+d ranging from 2 to 1000.

In an embodiment, the one or more treatment compositions comprises a solvent chosen from water, organic solvents, and a mixture thereof.

The agents and compositions for treating keratinous fibers, such as hair fibers, of the present invention includes amounts of each of the at least one polycarbodiimide compound, the at least one carboxylic acid, the at least one amine, and the at least one carboxysilicone polymer sufficient to impart to the keratinous fibers after application thereto, one or more of:

improved conditioning;
protection from damage caused by extrinsic and/or intrinsic factors;
repair when the keratinous fibers are in a damaged condition;
minimized or no breakage;
increased or improved mechanical strength;
increased hydrophobicity; or
increased hold to the shape or configuration of the fibers.

In an embodiment, the at least one of the one or more treatment compositions is a single-step treatment composition comprising:
the at least one carboxylic acid in an amount, by weight, of from about 0.05% to about 4%, or from about 0.1% to about 3%, based on the total weight of the composition;
the at least one amine is present in an amount, by weight, of from about 0.05% to about 4%, or from about 0.1% to about 3%, based on the total weight of the composition; and
the at least one polycarbodiimide compound in an amount, by weight, of from about 0.2% to about 5%, or from about 0.3% to about 3%, based on the total weight of the composition.

In an embodiment, the agent comprises two treatment compositions comprising:

(1) a first treatment composition containing:
the at least one carboxylic acid in an amount, by weight, of from about 0.05% to about 4%, or from about 0.1% to about 3%, based on the total weight of the first treatment composition;
the at least one amine is present in an amount, by weight, of from about 0.05% to about 4%, or from about 0.1% to about 3%, based on the total weight of the first treatment composition; and
the at least one polycarbodiimide compound in an amount, by weight, of from about 0.2% to about 5%, or from about 0.3% to about 3%, based on the total weight of the first treatment composition;
(2) a second treatment composition containing:
the at least one polycarbodiimide compound in an amount, by weight, of from about 0.2% to about 5%, or from about 0.3% to about 3%, based on the total weight of the second treatment composition; and
the at least one carboxysilicone polymer, in an amount, by weight, of from about 0.4% to about 10%, or from about 0.5% to about 5%, based on the total weight of the second treatment composition;
wherein the first and second treatment compositions are applied to the fibers in a step-wise manner in any order.

In an embodiment, the first and second treatment compositions above are applied to keratinous fibers according to a two-step application method comprising:

(i) applying the first treatment composition onto the fibers;
(ii) heating the fibers at a temperature above room temperature; and
(iii) applying the second treatment composition onto the fibers;
wherein the keratinous fibers include hair.

In an embodiment, the agent comprises three treatment compositions comprising:

(1) a first treatment composition containing:
the at least one carboxylic acid in an amount, by weight, of from about 0.05% to about 4%, or from about 0.1% to about 3%, based on the total weight of the first treatment composition; and
the at least one amine is present in an amount, by weight, of from about 0.05% to about 4%, or from about 0.1% to about 3%, based on the total weight of the first treatment composition; and
   (2) a second treatment composition containing the at least one polycarbodiimide compound in an amount, by weight, of from about 0.2% to about 5%, or from about 0.3% to about 3%, based on the total weight of the second treatment composition; and
   (3) a third treatment composition containing the at least one carboxysilicone polymer, in an amount, by weight, of from about 0.4% to about 10%, or from about 0.5% to about 5%, based on the total weight of the third treatment composition.

In an embodiment, the first, second, and third treatment compositions above are applied to keratinous fibers according to a three-step application method comprising:

(i) applying the first treatment composition of claim 16 onto the fibers;
(ii) leaving the first treatment composition on the fibers at room temperature for at least 30 minutes;
(iii) applying the second treatment composition of claim 16 onto the fibers;
(iv) applying the third treatment composition of claim 16 onto the fibers; and
(v) heating the fibers at a temperature above room temperature; wherein the keratinous fibers include hair.

In an embodiment, the first treatment composition as described above additionally contains at least one hair active agent chosen from oxidizing agents, reducing agents, neutralizing agents, dyeing agents, and mixtures thereof.

In an embodiment, present invention is directed to a method for treating keratinous fibers wherein the first treatment composition having at least one active agent chosen from oxidizing agents, reducing agents, neutralizing agents, dyeing agents, and mixtures thereof is applied onto the fibers, followed by (2) a second treatment composition comprising—the at least one polycarbodiimide compound in an amount, by weight, of from about 0.2% to about 5%, or from about 0.3% to about 3%, based on the total weight of the second treatment composition; and—the at least one carboxysilicone polymer, in an amount, by weight, of from about 0.4% to about 10%, or from about 0.5% to about 5%, based on the total weight of the third treatment composition.

After the application of the first treatment composition having at least one active agent, the method can include a step of applying onto the fibers a conditioning composition containing at least one conditioning agent chosen from cationic conditioning agents, silicone compounds, and mixtures thereof.

The cationic conditioning agents are preferably chosen from monoalkyl quaternary amines, dialkyl quaternary amines, amidoamines in cationic form, polyquarternium compounds, polydimethylsiloxanes, polydimethyl siloxanes having hydroxyl end groups, amino functional silicones, and mixtures thereof.

When the cationic agent is chosen from silicone compounds, the silicone compounds are preferably chosen from amino functional silicones chosen from amodimethicones and/or alkylamino silicones corresponding to formula (K) below:

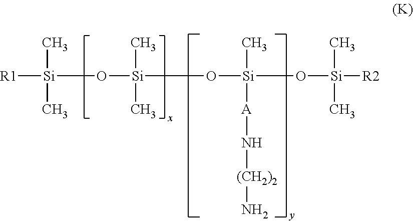

in which:
x and y are numbers ranging from 1 to 5000; preferably, x ranges from 10 to 2000 and especially from 100 to 1000; preferably, y ranges from 1 to 100;
R1 and R2, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms.

It has been surprisingly and unexpectedly discovered by the inventors that the treatment agents and methods of the present disclosure employing various compositions containing carboxylic acids chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, amines chosen from alkyl amines, alkanolamines, alkylene amines, and mixtures thereof, polycarbodiimide compounds, and carboxysilicone polymers in various combinations, when applied to keratinous fibers such as hair, enhance the properties of the substrates with respect to hydrophobicity, adhesion, chemical resistance, mechanical strength, cortex repair, and conditioning) and deliver superior performance to the fibers. In embodiments of this disclosure, the described methods, agents, and compositions enhance the properties of hair wherein the combination increases the conditioning effect (e.g., hydrophobicity, shine and smoothness), strengthens the hair (less or no breakage), increases the stiffness and humidity resistance of hair, ameliorates the condition of damaged hair by improving the appearance and quality of hair (for example, smoother feel, softer feel, less frizzy, less dry, more discipline) and repairs the hair cortex.

Without being bound to any one theory, the inventors of the present disclosure believe that the polycarbodiimide compound reacts with one or more of the carboxylic acids chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, the amines chosen from alkyl amines, alkanolamines, alkylene amines, and mixtures thereof, and the carboxysilicone polymers. It is also believed that these compounds not only react with each other and to the keratinous substrate via crosslinking reactions when such compositions are applied onto the substrates such as hair or skin. It is also believed that such reactions result into longer lasting or durable benefits or cosmetic attributes imparted to the hair.

The compositions according to the invention, are compositions including polycarbodiimide compounds, carboxylic acids chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof, amines chosen from alkyl amines, alkanolamines, alkylene amines, and mixtures thereof, and carboxysilicone polymers. The composition may include other suitable ingredients for hair treatment or hair repair. For example, known solvents and/or additives may be utilized in order to provide additional benefits to the composition. The range of concentrations by weight of the compositions over which the various associations of the polycarbodiimide, carboxylic acids, amines, and carboxysilicone polymers provides caring and/or repairing benefits to keratinous fibers is about 0.5% to about 40% by weight of total actives.

Carboxylic Acids

The at least one carboxylic acid of the present invention is chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and a mixture thereof.

The at least one carboxylic acid of the present invention will typically be present in the treatment compositions and agents of the present disclosure in an amount of from about 0.02% to about 5%, by weight, in some particular embodiments from about 0.05% to about 4%, by weight, and in some particular embodiments from about 0.1% to about 3%, by weight, including all ranges and subranges therebetween, based on the total weight of the treatment composition or agent.

In various embodiments, the amount of the at least one carboxylic acid in the treatment compositions and agents of the present disclosure is about 0.02%, 0.03%, 0.04%, 0.05%, 0.08%, 0.1%, 0.2%, 0.3%, 0.4%, 0.5%, 0.6%, 0.7%, 0.8%, 0.9%, 0.92%, 0.94%, 0.95%, 0.96%, 0.98%, 1%, 1.2%, 1.4%, 1.5%, 1.6%, 1.8%, 2%, 2.2%, 2.3%, 2.4%, 2.5%, 3%, 3.5%, 4%, 4.5%, and 5% by weight, based on the total weight of the treatment composition or agent.

Amines

The at least one amine of the present disclosure is chosen from alkylamines, alkanolamines, alkyleneamines, and mixtures thereof.

The at least one amine of the present disclosure is chosen from alkylamines, alkanolamines, alkyleneamines, and mixtures thereof may be chosen from amines comprising one or two primary, secondary, or tertiary amine functions, and at least one linear or branched C1-C8 alkyl groups bearing at least one hydroxyl radical.

In an embodiment, the at least one amine of the present disclosure may be chosen from any amine that has the formula R—CH2-NH2. By way of non-limiting example, amines useful according to various embodiments include 3-isopropoxypropylamine, 3-methoxypropylamine (3-MPA), tris(hydroxymethyl)aminomethane, 3-ethoxypropylamine, 3-(2-ethylhexyloxy)-propylamine, 2-(2-aminoethoxy)ethanol (2-2AEE), 3-butoxypropylamine (3-BPA), and monoethanolamine (MEA), and combinations thereof.

In an embodiment, the at least one amine of the present disclosure is chosen from alkanolamines such as mono-, di- or trialkanolamines, comprising one to three identical or different C1-C4 hydroxyalkyl radicals. Examples of alkanolamines that may be used in the compositions of the present disclosure are monoethanolamine (MEA), diethanolamine (DEA), triethanolamine (TEA), monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, 3-isopropoxypropylamine, 3-methoxypropylamine (3-MPA), tris(hydroxymethyl)aminomethane, and mixtures thereof.

In an embodiment, the at least one amine of the present disclosure is chosen from alkylamines such as ethylamines, and alkyleneamines such as ethyleneamines, including derivatives thereof and mixtures thereof.

In an embodiment, the at least one amine of the present disclosure is chosen from monoethanolamine (MEA), diethanolamine (DEA), triethanolamine (TEA), monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, 3-isopropoxypropylamine, 3-methoxypropylamine (3-MPA), tris(hydroxymethyl)aminomethane, 3-ethoxypropylamine, 3-(2-ethylhexyloxy)-propylamine, 2-(2-aminoethoxy)ethanol (2-2AEE), 3-butoxypropylamine (3-BPA), ethylamines, ethyleneamine, and mixtures thereof.

The at least one amine of the present invention will typically be present in the treatment compositions and agents of the present disclosure in an amount of from about 0.02% to about 5%, by weight, in some particular embodiments from about 0.05% to about 4%, by weight, and in some particular embodiments from about 0.1% to about 3%, by weight, including all ranges and subranges therebetween, based on the total weight of the treatment composition and agent.

In various embodiments, the amount of the amine in the treatment compositions and agents of the present disclosure is about 0.02%, 0.03%, 0.04%, 0.05%, 0.08%, 0.1%, 0.2%, 0.3%, 0.4%, 0.5%, 0.6%, 0.7%, 0.8%, 0.9%, 1%, 1.5%, 2%,2.5%, 3%, 3.5%, 4%, 4.5%, and 5% by weight, based on the total weight of the treatment composition and agent.

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

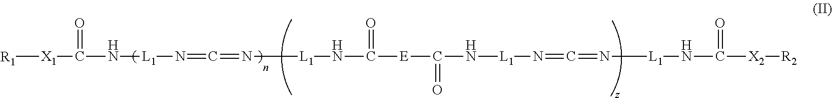

wherein X1 and X2 each independently represent O, S or NH. R1 and R2 are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L1 (Linker of carbodiimide groups) is selected from a C1 to C18 divalent aliphatic hydrocarbon group, a C3 to C13 divalent alicyclic hydrocarbon group, a C6 to C14 divalent aromatic hydrocarbon group, and a C3 to C12 divalent heterocyclic group; wherein a plurality of L1s may be identical to or different from one another, and wherein in another embodiment, L1 of formula (II) is selected from a C1 to C18 divalent aliphatic hydrocarbon group, a C3 to C13 divalent alicyclic hydrocarbon group, a C6 to C14 divalent aromatic hydrocarbon group, and a C3 to C12 divalent heterocyclic group; wherein a plurality of L1 s may be identical to or different from one another;

wherein E is a radical selected from the following formulas:

O—R3-O; S—R4-S; and

R5-N—R4-N—R5;

wherein R3 and R4 are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R5 is hydrogen, or a hydrocarbon radical which can contain halogen atoms or one or more catenary (i.e.; in chain, bonded only to carbon) or non-catenary hetero atoms.

Examples of R1 and R2 can be methyl glycolate, methyl lactate, polypropylene glycol, polyethylene glycol monomethyl ether, dialkylamino alcohol.

Examples of L1 can be the diradical of tolylene, hexamethylene, hydrogenated xylylene, xylylene, 2,2,4-trimethylhexamethylene, 1,12-dodecane, norbornane, 2,4-bis-(8-octyl)-1,3-dioctylcyclobutane, 4,4′-dicyclohexylmethane, tetramethylxylylene, isophorone, 1,5-naphthylene, 4, 4′ diphenylmethane, 4, 4′ diphenyldimethylmethane, phenylene.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

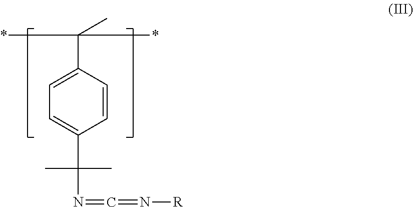

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

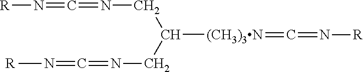

wherein R is an alkyl, cycloalkyl or aryl group (in some particular embodiments having from 1 to 24 carbon atoms).

An example of a polycarbodiimide compound that has a linker L1 chosen from m-tetramethylxylylene is an aromatic functionalized polycarbodiimide according to the following formula (V):

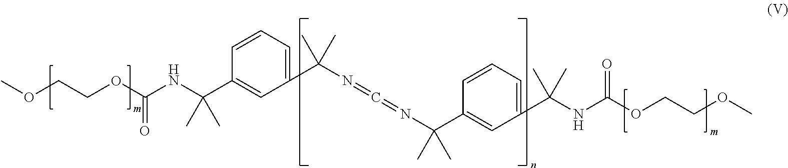

wherein m and n, each independently, are selected from integers from 1 to 100.

In one embodiment, the compositions of the present disclosure do not employ a polycarbodiimide compound of formula (V) above.

In one embodiment, the polycarbodiimide compound of the present disclosure is not chosen from the compound of formula (V) above.

Suitable polycarbodiimide compounds include, but are not limited to, those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide compounds of the present disclosure is selected from compounds of formula (II) wherein L1 (Linker of carbodiimide groups) represents a C1 to C18 divalent aliphatic hydrocarbon group, a C3 to C13 divalent alicyclic hydrocarbon group, a C3 to C12 divalent heterocyclic group, or a C6 to C14 divalent aromatic hydrocarbon group;

wherein a plurality of L1s may be identical to or different from one another.

The polycarbodiimide compound is typically present in the treatment compositions and agents of the present disclosure in an amount of from about 0.1% to about 10%, by weight, in some particular embodiments from about 0.2% to about 5%, by weight, or from about 0.3% to about 3%, by weight, including all ranges and subranges therebetween, based on the total weight of the treatment compositions and agents.

In various embodiments, the amount of the polycarbodiimide compound in the treatment compositions and agents of the present disclosure is about 0.1%, 0.2%, 0.25%, 0.3%, 0.35%, 0.4%, 0.42%, 0.44%, 0.45%, 0.42%, 0.46%, 0.48%, 0.5%, 0.55%, 0.6%, 0.65%, 0.7%, 0.75%, 0.8%, 0.85%, 0.9%, 0.95%, 0.975%, 1%, 1.1%, 1.2%, 1.4%, 1.5%, 2%, 2.5%, 3%, 3.5%, 4%, 4.5%, 5%, 5.5%, 6%, 6.5%, 7%, 7.5%, 8%, 8.5%, 9%, 9.5%, and 10%, by weight, based on the total weight of the treatment compositions and agents.

### Carboxysilicone Polymers

Silicone Polymers Having at Least One Carboxylic Acid Group (Carboxysilicone Polymers).

The silicone polymers having at least one carboxylic acid group, referred herein as carboxysilicone polymers, according to the present disclosure, may be an organopolysiloxane comprising:

(A) a compound having the following formula (VI):

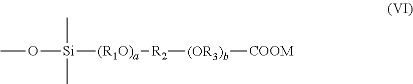

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or

(B) a group comprising at least one pyrrolidone carboxylic acid unit having the following formula (VII):

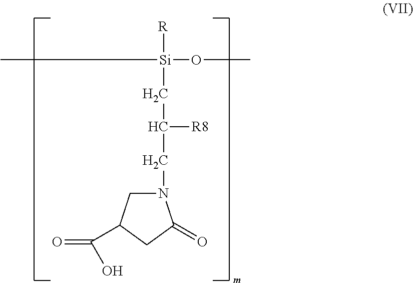

in which R is selected from methyl or phenyl; R8 is hydrogen or methyl, m is an integer from 1 to 1000, or

(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable carboxysilicone polymers include, for example, a silicone polymer comprising at least one carboxylic acid group chosen from organopolysiloxanes of formula (VIII):

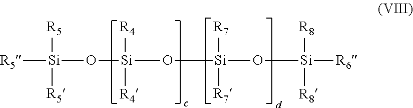

wherein the radicals R4, R4′ are identical to or different from each other and are chosen from a linear or branched C1-C22 alkyl radical, a C1-C22 alkoxy radical and a phenyl radical, the radicals R5, R5′, R5′, R6, R6′, R6″, R7, and R7′ are identical to or different from each other and are chosen from a linear or branched C1-C22 alkyl radical, a C1-C22 alkoxy radical, a phenyl radical, a radical —(R1O)a-R2-(OR3)b-COOM, a radical containing pyrrolidone carboxylic acid, a radical of polyvinyl acid/ester; and

wherein at least one of the radicals R5, R6 and R7 is a radical chosen from a radical —(R1O)a-R2-(OR3)b-COOM, a radical containing pyrrolidone carboxylic acid, a radical of polyvinyl acid/ester;

wherein R1, R2, R3, a, b and M have the same meaning as described in Unit (A) above;

wherein c and d are integers from 0 to 1000, the sum c+d in some particular embodiments ranging from 1 to 1000 or from 2 to 1000.

Among the carboxysilicone polymers of formula (VIII) that comprise at least one unit (VI), which in some particular embodiments are the compounds of formula (IX) below:

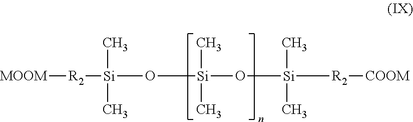

wherein R2, and M have the same meaning as described in Unit (A) above, n is an integer from 1 to 1000. Examples of compound (IX) are: dual-end carboxy silicones X-22-162C from Shin Etsu and Silform INX (INCI name: Bis-Carboxydecyl Dimethicone) from Momentive.

Other exemplary embodiments organopolysiloxanes of formula (VIII) are the ones of formula (X):

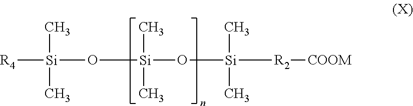

in which R₂, R₄, n, and M having the same meaning as in Unit (1) above. An example of compound (X) is a single-end carboxy silicone X-22-3710 from Shin Etsu.

Other exemplary embodiments organopolysiloxanes of formula (VIII) are the ones of formula (XI):

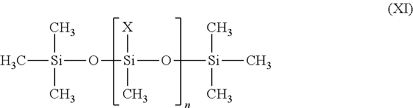

wherein X is a radical —(R1 O)a-R2-(OR3)b-COOM wherein R1, R2, R3, a, b and M have the same meaning as described in Unit (A) above.

Even more particularly, the compounds of formula (XI) in which a and b are equal to 0 and R₂ is a linear or branched C₂-C₁₂ alkylene group such as (CH₂)₉, (CH₂)₁₀ or —CH(CH₃)— are exemplary embodiments. An example of compound (XI) is a side-chain carboxy silicone X-22-3701E from Shin Etsu.

Among the organopolysiloxanes of formula (VIII) that contain unit (B), exemplary embodiments include the compounds of formula (XII) below:

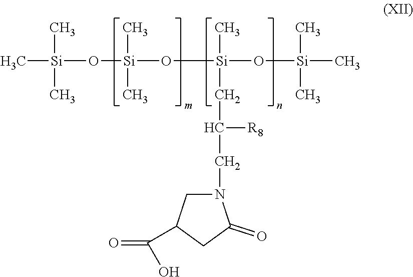

wherein R8, m, are defined as in Unit (B) above and n is an integer from 1 to 1000. An example of compound (XII) is Grandsil PCA such as in Grandsil SiW-PCA-10 (INCI name: Dimethicone (and) PCA Dimethicone (and) Butylene Glycol (and) Decyl Glucoside from Grant Industries.

Among the organopolysiloxanes of formula (VIII) that contain polyvinyl acid/ester Unit (C), exemplary embodiments are crosslinked anionic copolymers comprised of organic polymer blocks and silicone blocks, resulting in a multiblock polymer structure. In particular, the silicone-organic polymer compound of the present invention may be chosen from crosslinked anionic copolymers comprising at least one crosslinked polysiloxane structural unit. An example of such a branched multi-block carboxysilicone polymer is Belsil® P1101 (may also be known under the tradename Belsil® P1101) (INCI name: Crotonic Acid/Vinyl C8-12 Isoalkyl Esters/VA/Bis-Vinyldimethicone Crosspolymer, also known by the technical name of Crotonic Acid/Vinyl C8-12 Isoalkyl Esters/VA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Compounds corresponding to formula (XI) above are sold, for example, under the name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone.

Other non-limiting examples of carboxysilicone polymers are silicone carboxylate containing polymers (silicone carboxylates).

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the silicone carboxylate is a compound of formula (XIII):

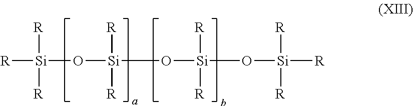

wherein a is an integer ranging from 1 to 100; b is an integer ranging from 0 to 500; and R, which may be identical or different, are each chosen from optionally substituted hydrocarbon groups comprising from 1 to 9 carbon atoms, optionally substituted phenyl groups, and groups of the following formula (XIV):

—(CH₂)₃—O-(EO)_{c}—(PO)_{d}—(EO)ₑ—C(O)—R′—C(O)—OH (XIV)

wherein c, d, and e, which may be identical or different, are each integers ranging from 0 to 20; EO is an ethylene oxide group; PO is a propylene oxide group; and R′ is chosen from optionally substituted divalent hydrocarbons, such as alkylene groups and alkenylene groups comprising from 2 to 22 carbon atoms, and optionally substituted divalent aromatic groups, such as groups of the following formula:

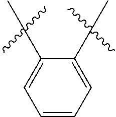 [SMILES: *c1ccccc1*]

and groups of the following formula:

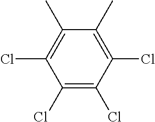

with the proviso that at least one of the R groups is chosen from groups of formula (XIV) and with the further proviso that when only one of the R groups is chosen from groups of formula (XIV), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (XV) below. Thus, in one embodiment, the at least one silicone carboxylate is chosen from a compound of formula (XV) and salts thereof:

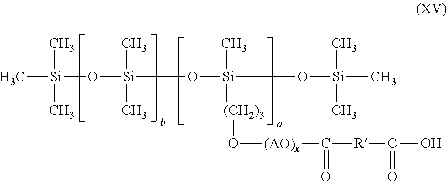

wherein a is an integer ranging from 1 to 100, b is an integer ranging from 0 to 500, AO is chosen from groups of the following formula:

-(EO)c-(PO)d-(EO)e-

wherein c, d, and e, which may be identical or different, are each integers ranging from 0 to 20; EO is an ethylene oxide group; PO is a propylene oxide group; x is an integer ranging from 0 to 60; R′ is chosen from optionally substituted divalent hydrocarbons, such as alkylene groups and alkenylene groups comprising from 2 to 22 carbon atoms, and optionally substituted divalent aromatic groups, such as groups of the following formula:

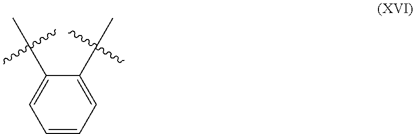 [SMILES: *c1ccccc1*]

and groups of formula

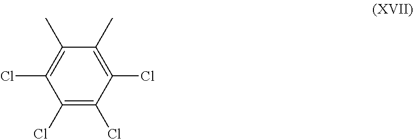

Non-limiting examples of silicone carboxylates include those described in U.S. Pat. Nos. 5,248,783 and 5,739,371, the disclosures of which are incorporated herein by reference, and which are silicone compounds of formula (XIII).

The carboxysilicone polymer will typically be present in the treatment compositions and agents of the present disclosure in an amount of from about 0.25% to about 20%, by weight, in some particular embodiments from about 0.5% to about 10%, by weight, and in some particular embodiments from about 0.5% to about 5.0%, by weight, including all ranges and subranges therebetween, based on the total weight of the treatment compositions and agents.

In various embodiments, the amount of the carboxysilicone polymer in the treatment compositions and agents of the present disclosure is about 0.25%, 0.5%, 0.55%, 0.6%, 0.65%, 0.7%, 0.75%, 0.8%, 0.85%, 0.9%, 0.925%, 0.95%, 0.975%, 1%, 1.1%, 1.2%, 1.3%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, 10%, 12%, 14%, 15%, 16%, 18%, and 20% by weight, based on the total weight of the treatment compositions and agents.

### Solvent

The composition, according to the present disclosure, further includes suitable solvents for treatment of keratinous fibers. Examples of suitable solvents include water, in some particular embodiments distilled or de-ionised, or organic solvents or mixtures thereof.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. In an exemplary embodiment, glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluoro oils such as nonafluoromethoxybutane and perfluoro methylcyclopentane.

Particularly suitable solvents in the composition of the present disclosure include water, isododecane, ethanol, and combinations thereof. The solvent will typically be present in total amounts ranging from about 60% to 98%, in some particular embodiments from 80% to 96%, by weight, including all ranges and subranges therebetween, based on the total weight of the composition.

In yet some other embodiments, the solvent of the present disclosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%,40%, 35%,30%, 20%, 10%, or 5% or less, by weight, based on the total weight of the composition.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%,40%, 35%,30%, 20%, 10%, or 5% or less, by weight, based on the total weight of the composition.

Hair Active Agents

The at least one hair active agent of the present disclosure may be chosen from oxidizing agents, reducing agents, dyeing agents, and mixtures thereof.

### Oxidizing Agents

Oxidizing agents may be selected from, for example, peroxides, persulfates, perborates percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. Oxygen in the air may also be employed as an oxidizing agent.

In some cases, the oxidizing agent is a persulfate and/or a monopersulfate such as, for example, potassium persulfate, sodium persulfate, ammonium persulfate, as well as mixtures thereof. In one embodiment, the oxidizing agents in the present disclosure are selected from hydrogen peroxide, potassium persulfate, sodium persulfate, and mixtures thereof.

One or more oxidizing agents are typically included in an oxidizing composition. An oxidizing composition may be a hair lightening or bleaching composition or it may be a neutralizing composition or a developer composition. In some cases, the total amount of the one or more oxidizing agents in an oxidizing composition is essentially 100% (as is the case for some powdered oxidation compositions). In some cases, the total amount of the one or more oxidizing agents is about 1 to about 80 wt. %, about 1 to about 70 wt. %, about 1 to about 60 wt. %, about 1 to about 50 wt. %, about 1 to about 40 wt. %, about 5 to about 80 wt. %, about 5 to about 70 wt. %, about 5 to about 60 wt. %, about 5 to about 50 wt. %, about 5 to about 40 wt. %, about 10 to about 80 wt. %, about 10 to about 70 wt. %, about 10 to about 60 wt. %, about 10 to about 50 wt. %, or about 10 to about 40 wt. %, based on the total weight of the composition.

The hair lightening compositions may be combined with a developer composition. A developer composition is typically an aqueous composition that includes one or more oxidizing agents, such as peroxide. Non-limiting examples of oxidizing include those that are water soluble such as peroxygen oxidizing agents. The oxidizing agent may be selected from water-soluble oxidizing agents which are inorganic peroxygen materials capable of yielding hydrogen peroxide in an aqueous solution.

The oxidizing agents may be provided in aqueous solution or as a powder which is dissolved prior to use. Water-soluble oxidizing agents include hydrogen peroxide, inorganic alkali metal peroxides such as sodium periodate and sodium peroxide and organic peroxides such as urea peroxide, and melamine peroxide. Alkyl and aryl peroxides, and or peroxidases may also be used. Other oxidizing agents such as inorganic perhydrate salts or persalts can be used as bleaching compounds or be present in bleach compositions, such as the alkali metal salts of perborates, percarbonates, perphosphates, persilicates, persulfates and the like. These inorganic perhydrate salts may be incorporated as monohydrates, tetrahydrates etc.

Mixtures of two or more such oxidizing agents can also be used if desired. In some cases, hydrogen peroxide, percarbonate, persulfates and combinations thereof, are used in the developer compositions.

Another potential oxidizing agent for use herein is a source of peroxymonocarbonate ions. Preferably such a source is formed in situ from a source of hydrogen peroxide and a hydrogen carbonate ion source. This system is particularly effective in combination with a source of ammonia or ammonium ions. Accordingly, any source of these peroxymonocarbonate ions may be utilized. Suitable sources for use herein include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. Percarbonate salts may also be utilized to provide both the source of carbonate ions and as an oxidizing agent. Non-limiting examples of carbonate ions, carbamate and hydrocarbonate ions are sodium hydrogen carbonate, potassium hydrogen carbonate, ammonium carbamate, and mixtures thereof.

The total amount of oxidizing agents in developer and bleach compositions can vary, but in some cases, can be from about 0.1 to about 50 wt. %, about 0.1 to about 40 wt. %, about 0.1 to about 30 wt. %, about 0.1 to about 25 wt. %, about 1 to about 50 wt. %, about 1 to about 40 wt. %, about 1 to about 30 wt. %, or about 1 to about 25 wt. %, based on the total weight of the composition.

### Reducing Agents

Reducing agents are well known for use in hair care compositions. Typical reducing agents are capable of reducing the disulfide bonds in the hair to produce free thiol groups. Non-limiting examples of suitable reducing agents include thioglycolic acid and thioglycolic acid salts and esters, thiolactic acid and thiolactic acid salts and esters, cysteine thioglycerol, thioglycolic hydrazide, thioglycolamide, glycerol monothioglycolate, sodium metabisulfite, beta-mercaptopropionic acid, N-hydroxyethyl mercapto-acetamide, N-methyl mercapto-acetamide, beta-mercapto-ethylamine, beta-mercaptopropionamide, 2-mercapto-ethanesulfonic acid, dimercaptoadipic acid, dithiothreitol, homocysteinethiolactone, cysteine derivatives, polythiol derivatives formed by the addition of cysteamine onto a maleic anhydride-alkylvinylether copolymer, inorganic sulfites, inorganic bisulfites, cysteamine and its derivatives, dithioerythritol, organic phosphines, and mixtures thereof.

One or more reducing agents may be included as hair active agents in the compositions of the present invention. The total amount of the one or more reducing agents can vary, but in some cases, the total amount of the one or more reducing agents is about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the composition.

### Neutralizing Agents

Neutralizing agents are well known for use in hair care compositions. For instance, the neutralizing agent may be an oxidizing agent chosen from hydrogen peroxide, urea hydrogen peroxide, alkali metal bromates, or persalts, such as perborates and persulfates. One or more neutralizing agents as hair active agents in the compositions of the present invention. The total amount of the one or more neutralizing agents can vary, but in some cases, the total amount of the one or more neutralizing agents is about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the composition.

### Colorants

Before, after, or simultaneously with the hair lightening composition or independently of hair lightening compositions, a coloring or dyeing composition may be used. For example, the coloring composition may be formed by combining a hair lightening composition according to the instant disclosure and a developer composition (typically comprising hydrogen peroxide) and a colorant. Typically, the coloring compositions include at least one colorant compound chosen from oxidative dye precursors, direct dyes, pigments, and mixtures thereof. The oxidation dyes are generally chosen from one or more oxidation bases optionally combined with one or more couplers. By way of example, the oxidation bases are chosen from para-phenylenediamines, bis(phenyl)alkylenediamines, para-aminophenols, ortho-aminophenols and heterocyclic bases, and the addition salts thereof.

Coloring compositions may optionally comprise one or more couplers advantageously chosen from those conventionally used in the dyeing or coloring of keratinous fibers.

Among these couplers, mention may be made especially of meta-phenylenediamines, meta-aminophenols, meta-diphenols, naphthalene-based couplers and heterocyclic couplers, and also the addition salts thereof.

In general, the addition salts of the oxidation bases and couplers that may be used in the context of the disclosure are especially selected from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the composition, and preferably from 0.005% to 5% by weight relative to the total weight of the compositions of the present disclosure.

The coupler(s), if they are present, each advantageously represent from 0.001% to 10% by weight relative to the total weight of the composition, and preferably from 0.005% to 5% by weight relative to the total weight of the compositions of the present disclosure.

Compositions according to the disclosure may optionally comprise b) one or more synthetic or natural direct dyes, chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Many direct dyes are cationic direct dyes. Mention may be made of the hydrazono cationic dyes and diazo cationic dyes

Among the natural direct dyes that may be used according to the disclosure, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the direct dye(s) more particularly represent from 0.001% to 10% by weight and preferably from 0.005% to 5% by weight of the total weight of the compositions of the present disclosure.

Conditioning Agents and Conditioning Compositions

The compositions and agents for treating keratinous fibers in accordance with the present disclosure may comprise conditioning compositions or conditioning agents that include cationic conditioning agents, silicone compounds, and mixtures thereof.

### Cationic Conditioning Agents

The cationic conditioning agent employed in the compositions of the present invention can be a monoalkyl quaternary amine, such as stearyltrimonium chloride, soyatrimonium chloride or coco-ethyldimonium ethosulfate behentrimonium chloride, cetrimonium chloride, and mixtures thereof. Other suitable cationic conditioning agents include, but are not limited to, dialkyl quaternary amines, such as dicetyldimonium chloride, dicocodimethyl ammonium chloride or distearyldimethyl ammonium chloride; polyquaternium compounds, such as Polyquaternium-6, Polyquaternium-22 or Polyquaternium-5; and amidoamines in cationic form (depending on pH or when neutralized), such as stearamidopropyl dimethylamine or brassicamidopropyl dimethylamine; or mixtures thereof.

The cationic conditioning agent employed in the compositions of the present invention is a non-silicone-based cationic conditioning agent.

In some instances, the cationic conditioning agents are cationic conditioning polymers. Examples of cationic conditioning polymers that can be used include, without limitation, cationic cellulose, cationic proteins, and cationic polymers. The cationic polymers can have a vinyl group backbone of amino and/or quaternary ammonium monomers. Cationic amino and quaternary ammonium monomers include, without limitation, dialkylamino alkylmethacrylate, monoalkylaminoalkyl acrylate, monoalkylaminoalkyl methacrylate, trialkyl methacryoloxyalkyl ammonium salt, trialkyl acryloxyalkyl ammonium salts, diallyl quaternary ammonium salts, vinyl compounds substituted with dialkyl aminoalkyl acrylate, and vinyl quaternary ammonium monomers having cyclic cationic nitrogen containing rings such as pyridinium, imidazolium, or quaternized pyrrolidine. Other examples of cationic conditioning polymers that can be used include, without limitation, hydroxypropyltrimonium honey, cocodimonium silk amino acids, cocodimonium hydroxypropyl hydrolyzed wheat or silk protein, polyquaternium-5, polyquaternium-11, polyquaternium-2, polyquaternium-4, polyquaternium-6, polyquaternium-7, polyquaternium-7, polyquaternium-14, polyquaternium-16, polyquaternium-22, polyquaternium-10, and guar hydroxypropyltrimonium chloride.

In some cases quaternized polymeric cationic conditioning agents are particularly useful. Particularly preferred are quaternary nitrogen polymers prepared by the polymerization of a dialkyldiallylammonium salt or copolymer thereof in which the alkyl group contains 1 to about 18 carbon atoms, and more preferably where the alkyl group is methyl or ethyl. Details concerning the preparation of these polymers can be found in U.S. Pat. Nos. 3,288,770, 3,412,019 and 4,772,462, incorporated herein by reference. For example, cationic homopolymers and copolymers of polydiallyldimethylammonium chloride are available in aqueous compositions sold under the trademark MERQUAT by the Calgon Corporation, subsidiary of Merck & Co., Pittsburgh, Pa. The homopolymer, which has the INCI name of Polyquaternium-6 is available under the trademark MERQUAT-100, and is described as having a weight average molecular weight of approximately 100,000. A copolymer reaction product of dimethyldiallylammonium chloride with acrylamide monomers is named Polyquaternium-7 in the CTFA Dictionary, is described as having a weight average molecular weight of approximately 500,000 and is sold under the trademark MERQUAT-550. Another copolymer reaction product of dimethyldiallylammonium chloride with acrylic acids having a weight average molecular weight from about 50,000 to about 10,000,000 has the CTFA name Polyquaternium-22 and is sold under the trademark MERQUAT-280. Polyquaternium-6 is particularly preferred.

Other polymeric conditioners include cationic copolymers of methylvinylimidazolium chloride and vinyl pyrrolidone, sold commercially by BASF Aktiengesellschaft, West Germany under the trademark LUVIQUAT at three comonomer ratios, namely at ratios of 95/5, 50/50 and 30/70 methylvinylimidazolium chloride to polyvinylpyrrolidone. These copolymers at all three comonomer ratios have the CTFA name Polyquaternium 16. Polymeric conditioners also include cationic cellulosic polymers of hydroxyethyl cellulose reacted with epichlorohydrin and quaternized with trimethylamine, sold under the trademark POLYMER JR in various viscosity grades and molecular sizes by Union Carbide Corporation, Danbury, Conn. These series of polymers are named Polyquaternium 10 in the CTFA Dictionary. Also useful are quaternized copolymers of hydroxyethylcellulose and dimethyldimethylammonium chloride, having the CTFA name Polyquaternium-4, sold in varying molecular weights under the trademark CELQUAT by National Starch and Chemical Corporation, Bridgewater, N.J.

Smaller molecule cationic non-polymeric conditioning agents can also be utilized herein. Exemplary small-molecule conditioning agents can include monofunctional or difunctional quaternary ammonium compounds, such as stearyldimethylbenzylammonium chloride, dimethyldi-(hydrogenated tallow)ammonium chloride, and the like. Non-polymeric conditioning agents can also include the quaternary ammonium salts of gluconamide derivatives, such as gamma-gluconamidopropyldimethyl-2-hydroxyethyl-ammonium chloride and minkamidopropyldimethyl-2-hydroxyethylammonium chloride identified respectively by the CTFA names Quaternium 22 and Quaternium 26. Details for the preparation of these materials are found in U.S. Pat. Nos. 3,766,267 and 4,012,398, respectively, and the materials are sold under the trademark CERAPHYL by Van Dyk & Co., Belleville, N.J. Also useful are bis-quaternary ammonium compounds which are dimers, such as 2-hydroxy propylene-bis-1,3-(dimethylstearyl ammonium chloride, designated the CTFA name, Hydroxypropyl Bisstearyldimonium chloride. The preparation of these and other bis-quat materials is described in U.S. Pat. No. 4,734,277, and such materials are sold under the trademark JORDAQUAT DIMER by Jordan Chemical Company, Folcroft, Pa.

Exemplary unquaternized polymers having tertiary amino nitrogen groups that become quaternized when protonated can include water-soluble proteinaceous quaternary ammonium compounds. Cocodimonium hydrolyzed animal protein, for example, is the CTFA name for a chemically-modified quaternary ammonium derivative of hydrolyzed collagen protein having from about 12 to about 18 carbons in at least one aliphatic alkyl group, a weight average molecular weight from about 2500 to about 12,000, and an isoionic point in a range from about 9.5 to about 11.5. This material and structurally related materials are sold under the trademarks CROQUAT and CROTEIN by Croda, Inc., New York, N.Y.

In an embodiment, cationic conditioning agents may be chosen from polyquarternium-10 (also called quaternized polyhydroxyethyl cellulose), cetrimonium chloride (also called cetyl trimethyl ammonium chloride, CTAC), behentrimonium chloride (also known as docosyl trimethyl ammonium chloride or BETAC), behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, dilinoleamidopropyl dimethylamine dimethicone PEG-7 phosphate, and mixtures thereof.

### Silicone Compounds

The silicone compounds that can be used as conditioning agents in the hair treatment compositions of the inventions of the present disclosure include polydiorganosiloxanes, in particular polydimethylsiloxanes (INCI name is dimethicone.) and polydimethyl siloxanes having hydroxyl end groups (INCI name is dimethiconol). Other examples are silicone gums having a slight degree of cross-linking.

Other suitable examples of the silicone compounds of the present disclosure can comprise emulsified particles of silicone. These emulsified particles of silicone are insoluble in the aqueous matrix of the composition, that is, the silicone is present as dispersed particles.

The viscosity of the emulsified silicone itself (not the emulsion or the final conditioning composition) is typically at least 10,000 cst., or at least 60,000 cst, or at least 500,000 cst, or at least 1,000,000 cst., and preferably not exceeding 109 cst.

Emulsified silicones for use in the conditioning composition of the invention will typically have an average silicone particle size in the composition of less than 30, preferably less than 20, more preferably less than 10 microns. For good conditioning performance, the average silicone particle size of the emulsified silicone in the composition is less than 2 microns, ideally it ranges from 0.01 to 1 micron (generally called microemulsions).

Particle size may be measured by means of a laser light scattering technique, using a 2600D Particle Sizer from Malvern Instruments.

Suitable silicone emulsions for use in the invention are also commercially available in a pre-emulsified form.

Examples of suitable pre-formed emulsions include emulsions DC2-1766, DC2-1784, and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

Another group of silicones that may be employed in conditioner compositions of the invention are amino functional silicones. By “amino functional silicone” is meant a silicone containing at least one primary, secondary or tertiary amine group, or a quaternary ammonium group.

As amino silicone that may be used in the scope of the invention, the following can be cited:

a) Polysiloxanes Corresponding to Formula (A):

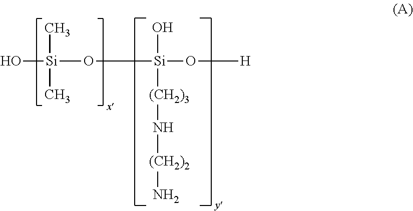

in which x′ and y′ are integers such that the weight-average molecular weight (Mw) is comprised between about 5000 and 500 000;

b) Amino Silicones Corresponding to Formula (B):

R′aG3-a-Si(OSiG2)n-(OSiGbR′2-b)m-O-SiG3-a-R′a (B)

in which:
G, which may be identical or different, designate a hydrogen atom, or a phenyl, OH or C1-C8 alkyl group, for example methyl, or C1-C8 alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH2qL in which q is a number ranging from 2 to 8 and L is an optionally quaternized amino group chosen from the following groups:
—NR″-Q-N(R″)2
—N(R″)2
—N+(R″)3A-
—N+H(R″)2A-
—N+H2(R″) A-
—N(R″)-Q-N+R″H2A-
—NR″-Q-N+(R″)2H A-
—NR″-Q-N+(R″)3A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, preferably from 2 to 4; and A—represents a cosmetically acceptable ion, in particular a halide such as fluoride, chloride, bromide or iodide.

A group of amino silicones corresponding to this definition (B) is represented by the silicones called “trimethylsilylamodimethicone” having formula (C):

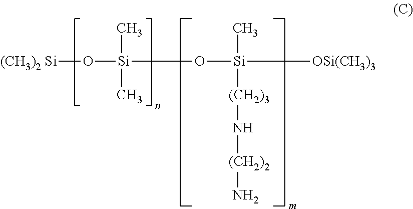

in which n and m have the meanings given above, in formula B.

Another group of amino silicones corresponding to this definition is represented by silicones having the following formulae (D) or (E):

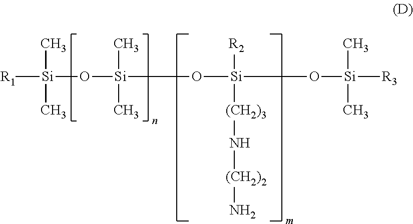

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R1, R2, R3, which may be identical or different, represent a hydroxy or C1-C4 alkoxy radical, where at least one of the radicals R1 to R3 denotes an alkoxy radical.

The alkoxy radical is preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

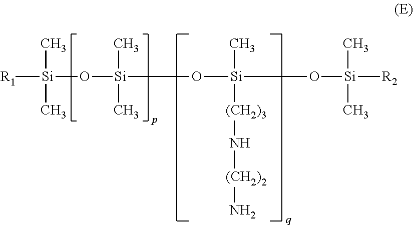

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R1, R2, which are different, represent a hydroxy or C1-C4 alkoxy radical, where at least one of the radicals R1 or R2 denotes an alkoxy radical.

The alkoxy radical is preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name BELSIL ADM 652.

A product containing amino silicones having structure (E) is sold by Wacker under the name FLUID WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino silicones having formula (E), microemulsions are used whose average particle size ranges from 5 nm to 60 nanometres (limits included) and more preferably from 10 nm to 50 nanometres (limits included). Accordingly, according to the invention the microemulsions of amino silicone having formula (E) sold as Finish CT 96 E® or SLM 28020® by Wacker can be used.

Another group of amino silicones corresponding to this definition is represented by the following formula (F):

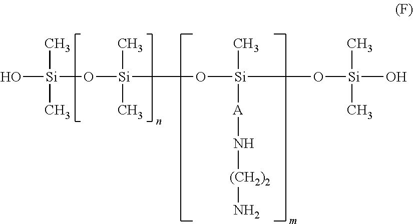

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

A preferred silicone of formula (F) is amodimethicone (INCI name) which is available under the tradename XIAMETER® MEM-8299 Cationic Emulsion by Dow Corning.

Another group of amino silicones corresponding to this definition is represented by the following formula (G):

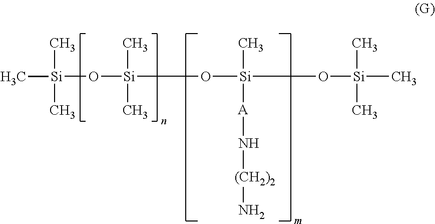

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

A silicone having this formula is for example DC2-8566 Amino Fluid by Dow Corning.

c) Amino Silicones Corresponding to Formula (H):

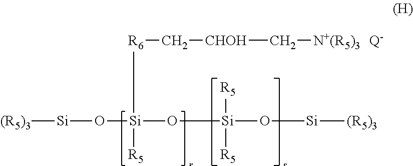

in which:
R5 represents a monovalent hydrocarbon-based radical containing from 1 to 18 carbon atoms, and in particular a C1-C18 alkyl or C2-C18 alkenyl radical, for example methyl;
R6 represents a divalent hydrocarbon-based radical, in particular a C1-C18 alkylene radical or a divalent C1-C18, for example C1-C8, alkylenoxy radical linked to the Si via an SiC bond;
Q—is an anion such as a halide ion, in particular chloride, or an organic acid salt (for example acetate);
r represents a mean statistical value from 2 to 20 and in particular from 2 to 8:
s represents a mean statistical value from 20 to 200 and in particular from 20 to 50.

d) Quaternary Ammonium Silicones Having Formula (I):

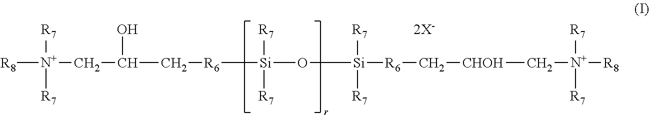

in which:
R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;
R6 represents a divalent hydrocarbon-based radical, in particular a C1-C18 alkylene radical or a divalent C1-C18, for example C1-C8, alkylenoxy radical linked to the Si via an SiC bond;
R8, which may be identical or different, represent a hydrogen atom, a monovalent hydrocarbon-based radical containing from 1 to 18 carbon atoms, and in particular a C1-C18 alkyl radical, a C2-C18 alkenyl radical or a —R6-NHCOR7 radical;
X— is an anion such as a halide ion, in particular chloride, or an organic acid salt (for example acetate);
r represents a mean statistical value from 2 to 200 and in particular from 5 to 100;

e) Amino Silicones Having Formula (J):

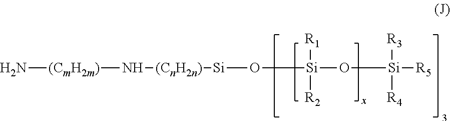

in which:
R1, R2, R3 and R4, which may be identical or different, denote a C1-C4 alkyl radical or a phenyl group;
R5 denotes a C1-C4 alkyl radical or a hydroxyl group;
n is an integer ranging from 1 to 5;
m is an integer ranging from 1 to 5;

and in which x is chosen such that the amine number is between 0.01 and 1 meq/g;

f) multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe2O)xSiMe2-R—N(R″)—R′—O(C2H4O)a(C3H6O)b-R′—N(H)—R—]

or alternatively

[—(SiMe2O)xSiMe2-R—N(R″)—R′—O(C2H4O)a(C3H6O)b-]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C2-C12 hydrocarbon-based radical, optionally including one or more heteroatoms such as oxygen; preferably, R denotes an ethylene radical, a linear or branched propylene radical, a linear or branched butylene radical, or a CH2CH2CH2OCH(OH)CH2- radical; preferentially R denotes a —CH2CH2CH2OCH(OH)CH2- radical;
R′, which may be identical or different, represent a divalent linear or branched C2-C12 hydrocarbon-based radical, optionally including one or more heteroatoms such as oxygen; preferably, R′ denotes an ethylene radical, a linear or branched propylene radical, a linear or branched butylene radical, or a —CH2CH2CH2OCH(OH)CH2- radical; preferentially R′ denotes —CH(CH3)-CH2-.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names SILSOFT A-843 or SILSOFT A+ by Momentive.

g) The Alkylamino Silicones Corresponding to Formula (K) Below:

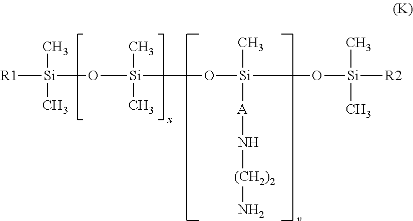

in which:
x and y are numbers ranging from 1 to 5000; preferably, x ranges from 10 to 2000 and especially from 100 to 1000; preferably, y ranges from 1 to 100;
R1 and R2, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms.

Preferably, A comprises 3 to 6 carbon atoms, especially 4 carbon atoms; preferably, A is branched. Mention may be made especially of the following divalent radicals: —CH2CH2CH2 and —CH2CH(CH3)CH2-.

Preferably, R1 and R2, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:
x ranging from 10 to 2000 and especially from 100 to 1000;
y ranging from 1 to 100;
A comprising 3 to 6 carbon atoms and especially 4 carbon atoms; preferably, A is branched; and more particularly A is chosen from the following divalent radicals: CH2CH2CH2 and —CH2CH(CH3)CH2-; and
R1 and R2, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A preferred silicone of formula (K) is bis-cetearylamodimethicone (INCI name).

Mention may be made especially of the silicone sold under the name Silsoft™ AX by Momentive.

Preferably, the amino silicones according to the invention are chosen from the amino silicones of formula (F). A preferred silicone of formula (F) is amodimethicone (INCI name) sold under the tradename XIAMETER® MEM-8299 Cationic Emulsion by Dow Corning.

The total amount of the one or more conditioning agents in the conditioning composition, may vary. In some cases, the total amount of the one or more conditioning agents is from about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the conditioning composition.

In some embodiments, the total amount of the one or more conditioning agents in the conditioning composition, is in an amount of from about 0.1 to about 5% by weight, about 0.2 to about 4% by weight, about 0.4 to about 3% by weight, about 0.5 to about 2% by weight, or about 0.5 to about 1% by weight, based on the total weight of the conditioning composition, including all ranges and sub ranges therebetween. In a particular embodiment, the amount of the silicone compound is at about 0.4%, 0.43%, 045%, 0.5%, 0.55%, 0.57%, 0.6%, 0.65%, 0.7%, 0.75%, 0.78%, 0.8%, 0.85%, 0.9%, 0.95%, 1%, 1.1% 1.2%, 1.3%, 1.4% 1.5%, 1.6%, 1.7%, 1.8% 1.9%, 2%, 2.1%, 2.2% 2.3%, 0.2.4% 2.5%, 2.6%, 2.7%, 2.8% 2.9%, 3%, 3.1% 3.2%, 3.3%, 3.4% 3.5%, 3.6%, 3.7%, 3.8% 3.9%, 4% by weight, based on the total weight of the conditioning composition.

Additives

The composition, according to the present disclosure, further includes suitable additives for treatment of keratinous fibers.

The composition according to the disclosure may also comprise additives chosen from surfactants (anionic, nonionic, cationic and amphoteric/zwitterionic), rheology modifiers, thickening and/or viscosity modifying agents, nacreous agents, opacifiers, fragrances, vitamins, preserving agents, pH stabilizers, and mixtures thereof.

The compositions of certain embodiments may comprise stabilizers, for example sodium chloride, magnesium dichloride or magnesium sulfate.

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods of Preparation and Methods of Use

In some embodiments, the compositions of the present disclosure are prepared by combining the at least one polycarbodiimide compound, the at least one carboxylic acid, and the at least one amine, and a solvent chosen from water, organic solvents, and mixtures thereof.

One-Step Application Process

In one embodiment, the composition of the present disclosure is applied onto keratinous fibers such as hair in a one-step application process. The composition for use in the one-step application is prepared by combining the ingredients the at least one polycarbodiimide compound, the at least one carboxylic acid, and optionally, the at least one amine, and a solvent, resulting in a treatment composition or agent that is then applied onto the keratinous fibers.

Two-Step Application Process

In other embodiments of the present disclosure, keratinous fibers such as hair, are treated in a two-step application process, i.e., the fibers are treated by an agent comprised of two treatment compositions. Thus, in one embodiment of the two-step application process, two treatment compositions are applied to the keratinous fibers in a step-wise fashion in any order to treat the substrate, wherein a first treatment composition contains the at least one polycarbodiimide compound, the at least one carboxylic acid, and the at least one amine, and a second treatment composition contains the at least one polycarbodiimide compound and the at least one carboxysilicone polymer.

In an embodiment, the first treatment composition is a heat-activated composition such that when applied onto the fibers, the treated fibers are heated at a temperature above room temperature, and then the second treatment composition is applied onto the heated fibers.

Three-Step Application Process

In other embodiments of the present disclosure, keratinous fibers such as hair, are treated in a three-step application process i.e., the fibers are treated by an agent comprised of three treatment compositions. Thus, in one embodiment of the three-step application process, three treatment compositions are applied to the keratinous fibers in a step-wise fashion in any order to treat the substrate, wherein a first treatment composition contains the at least one carboxylic acid, and the at least one amine, a second treatment composition contains the at least one polycarbodiimide, a third treatment composition contains the at least one carboxysilicone polymer. Preferably, the first treatment composition is applied first onto the fibers, then the second treatment composition is applied onto the fibers, and then the third treatment composition is applied onto the fibers. The fibers are then heated are heated at a temperature above room temperature.

The keratinous fibers may be heated by applying heat to the hair or exposing hair to elevated temperatures or the temperatures above room temperature; the temperature of heating can be equal to or above 30° C., or above 40° C. or above 50° C., or above 60° C., or above 70° C., or above 80° C., or above 90° C., or above 100° C. While not so limited, heating may be provided, for example, by commonly used heating tools for example a helmet dryer or blow dryer (about 30° C. and above, such as from about 30° C. to about 100° C. or from about 4000° C. to about 80° C. or from about 40° C. to about 70° C.) or hot iron or flat iron (about 130° C.-about 250° C.) or steam/hot rollers.

Chemical Treatment of Keratinous Fibers

In an embodiment of the present disclosure, keratinous fibers such as hair, are chemically treated in a two-step application process or a three-step application process.

In one embodiment of the two-step application process involving a chemical treatment, two treatment compositions are applied to the keratinous fibers in a step-wise fashion, wherein the first treatment composition contains the at least one carboxylic acid, the at least one amine, and a hair active agent, and the second composition contains the at least one polycarbodiimide compound and the at least one carboxysilicone polymer. A rinsing step is employed between the two applications.

In one embodiment of the three-step application process involving a chemical treatment, three compositions are applied to the keratinous fibers in a step-wise fashion, wherein the first composition is a treatment composition containing the at least one carboxylic acid, the at least one amine, and a hair active agent, the second composition is a conditioner composition containing a conditioning agent chosen from a cationic conditioning agent, a silicone compound, and mixtures thereof, the third composition is a treatment composition containing the at least one polycarbodiimide compound and the at least one carboxysilicone polymer. A rinsing step is employed after the application of the conditioning composition.

The hair active agent is chosen from oxidizing agents, reducing agents, dyeing agents, neutralizing agents, and mixtures thereof

The compositions and methods, according to the present disclosure, provides advantageous properties to keratinous fibers. In one embodiment, the compositions and methods, according to the present disclosure, provide hydrophobicity or impart hydrophobicity to hydrophilic or damaged keratinous fibers, such as damaged hair (bleached), upon application thereto. In certain embodiments, hydrophobicity is provided at room temperature, i.e., without heating or applying heat to the hair. In other embodiments, the hydrophobicity is provided when heat is used on the hair (before or after applying the composition on the hair or during the application of the composition on the hair).

In one embodiment, the hydrophobicity provided to less hydrophobic or to hydrophilic keratinous fibers includes a contact angle of greater than 50⁰ or greater than 60° or greater than 70° or greater than 80° or greater than 90° or greater than 100° or greater than 110°.

Further disclosed herein is the use of the compositions of the present disclosure for caring for keratinous fibers, for example, hair, such as for hair repair treatments, or for reducing damage to the hair or for imparting improved strength to hair or reducing/preventing hair breakage.

The compositions of the present disclosure may be employed in an effective amount to adequately cover the surface of the fibers of the hair and to achieve the desired effects of hair repair and conditioning.

An effective amount of the composition is typically from about 0.1 gram to about 50 grams per head of hair, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous substrates, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

Further disclosed herein is the use of the compositions of the present disclosure for shaping or styling hair and/or retaining a hairstyle. Also disclosed is the use of the compositions of the present disclosure for caring for the hair such as for hair repair treatments, or for reducing damage to the hair or for improving the feel of the hair by imparting hydrophobicity to the hair. The compositions may be applied to wet or dry hair. They may be used in a non-rinse fashion. In some other embodiments, the composition may be rinsed from the hair.

In an embodiment, the hair that has been contacted with the treatment compositions and agents of the present invention is further heated or exposed to elevated temperatures (above room temperature). The heat source can be chosen from a blow dryer, a flat iron, a hair dryer, a heat lamp, a heat wand, or other similar devices.

A smoothing or crimping action may be applied on the hair while heating the hair by use of suitable devices, including a hair brush, comb, or flat iron. The smoothing action on the hair may also include running the fingers through the hair.

A suitable applicator device for contacting the hair with compositions of the present invention is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes. In alternate embodiments, the treatment times may be longer, and in some embodiments, appreciably longer, such that the application may be left on for up to 24 hours to about 48 hours.

The compositions of the present invention are easy to spread on hair.

It has surprisingly and unexpectedly discovered that the application of the composition onto hair results in improved mechanical strength or repaired split ends, or more conditioned hair or of making the hair more humidity resistant. It was also surprisingly and unexpectedly discovered that the application of the composition of the present invention onto the hair resulted in improved hair quality, for example, one or more of better hair feel, less damaged feel, smoother hair, better hair manageability, or improved appearance of the hair (e.g., healthy look).

The composition, according to the present disclosure, when applied to hair, provides a strengthening and repair benefit to the hair which can be determined from tensile testing of the hair fibers based on the parameters of plateau stress, break stress, toughness, and number of cycles to break values. Increases in these values indicate that the hair fiber is stronger or became stronger/reinforced (requires more force to break) when treated according to the compositions, agents, and methods of the present invention.

In one embodiment, the plateau stress on hair fibers treated according to the present invention is equal to or greater than 100 MPa or equal to or greater than 105 MPa and can range from about 100 to about 200 MPa, including all ranges and sub-ranges therebetween. In one embodiment, the plateau stress values can be in the range of about 100 to about 140 MPa or about 105 to about 135 MPa or about 108 to about 135 MPa, including all ranges and sub-ranges therebetween. In an embodiment involving the two-step or the three-step application process, the plateau stress values can be in the range of about 110 to about 200 MPa or about 120 to about 195 MPa or about 123 to about 190 MPa, or about 135 to about 190 MPa, including all ranges and sub-ranges therebetween.

In one embodiment, the break stress on hair fibers treated according to the compositions and the methods of the present invention is equal to or greater than 120 MPa or equal to or greater than 130 MPa and can range from about 120 to about 300 MPa, including all ranges and sub-ranges therebetween. In one embodiment, the break stress values can be in the range of about 100 to about 220 MPa or about 120 to about 215 MPa or about 130 to about 210 MPa, including all ranges and sub-ranges therebetween. In an embodiment involving the two-step or the three-step application process, the break stress values can be in the range of about 170 to about 300 MPa or about 180 to about 295 MPa or about 185 to about 290 MPa, or about 187 to about 290 MPa, including all ranges and sub-ranges therebetween.

In one embodiment, toughness of hair fibers treated according to the compositions and the methods of the present invention is equal to or greater than 3 10⁷ J/m³ or equal to or greater than 5 10⁷ J/m³ and can range from about 3 to about 12 10⁷ J/m³, including all ranges and sub-ranges therebetween. In one embodiment, the toughness values can range from about 3 to about 9 10⁷ J/m³ or from about 3.2 to about 8.5 10⁷ J/m³ or from about 3.4 to about 8 10 J/m³, including all ranges and sub-ranges therebetween. In an embodiment involving the two-step or the three-step application process, the toughness values can range from about 4 to about 11 10⁷ J/m³ or from about 4.5 to about 10 10⁷ J/m³ or from about 5 to about 10 10⁷ J/m³, including all ranges and sub-ranges therebetween.

In one embodiment, the number of cycles to break of hair fibers treated according to the compositions and the methods of the present invention is equal to or greater than 2000 or equal to or greater 2500 or equal to or greater 8000 and can range from about 2000 to about 15,000, including all ranges and sub-ranges therebetween. In one embodiment, the toughness values can range from about 2000 to about 5000 or from about 2500 to about 5000 or from about 2800 to about 4500, including all ranges and sub-ranges therebetween. In an embodiment involving the two-step or the three-step application process, the toughness values can range from about 8000 to about 15,000 or from about 9000 to about 14,000 or from about 9500 to about 13,800, including all ranges and sub-ranges therebetween.

The hair care and hair repair effects obtained using the compositions and methods of the present invention may also be durable or long-lasting, i.e., wash or shampoo resistant.

As used herein, “long-lasting” or “durable” is understood to mean that the benefits imparted to hair by the compositions of the invention last over a period of time and/or over high humidity conditions and/or after one or multiple wash cycles (with water or shampoo/water or shampoo/water/conditioner/water or conditioner/water). The multiple wash cycles is understood to mean one or more wash cycles, such as one or two or three or four or five or six or seven or eight or nine or ten wash cycles.

Instructions for applying the composition of the present invention onto keratinous substrates such as hair on the head or eyelashes or skin may comprise directions of use of the composition for the end-user to follow. The end-user may be a consumer or cosmetologist or salon hair dresser. Directions may comprise instructing the end-user to take an amount of the composition in sufficient quantity such that the composition adequately covers the substrates and imparts the desired effects. Directions may additionally instruct the end-user to use a device (e.g., heating device) to achieve the desired effects.

Instructions for using the composition(s) of the present invention may appear on the container (such as can, bottle or jar) holding the composition(s) of the present invention or on the box or carton or other packaging comprising the container(s) holding the composition(s).

The compositions described above are useful for application onto keratinous fibers such as hair on the head of human individuals.

Thus, the compositions of the present invention can be made into various cosmetic products such hair care hair treatment, and hair styling products.

Representative types of hair care compositions, including hair care and styling compositions, of the present invention include compositions for conditioning or protecting hair from heat damage, leave-in hair treatments, rinse-off hair treatments, combination shampoo/styling compositions, hair volumizing compositions, compositions for shaping the hair or maintaining the shape of the hair or styling products (e.g., gels, creams, milks, pastes, waxes, ointments, serums, foams, hair lotions, mousses, pump-sprays, non-aerosol sprays and aerosol sprays).

The compositions of the present invention can be in the form of an aqueous composition or an emulsion, such as a lotion or cream, and in some embodiments may be applied in another form, such as in a serum such as an anhydrous serum (substantially free of water, for example containing less than 0.1% water or not containing added water to the composition).

In one embodiment, the composition of the present invention is in the form of a cream.

The compositions may be packaged in various forms, especially in a tube, a jar or bottles, in pump bottles, in squeeze bottles, or in aerosol containers so as to apply the composition in vaporized form or in the form of a mousse. The compositions may also impregnate applicators, especially gloves or wipes.

The composition may be applied by hand, with an applicator nozzle or actuator pump, with a container equipped with a pump, an applicator and a dispensing comb, or with an insoluble substrate impregnated with the composition.

As used herein, the methods and compositions disclosed herein may be used on the hair that has not been artificially dyed, pigmented or permed.

As used herein, the methods and compositions disclosed herein may be also used on the hair that has been artificially dyed, pigmented or permed, relaxed, straightened or other chemical process.

As used herein, the methods and compositions disclosed herein may be also used on the hair that is undergoing a chemical treatment such as a bleaching or lightening treatment or an oxidative dyeing treatment or a relaxing/straightening treatment or a perming/waving treatment.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

### EXAMPLES

The following examples are to illustrate the invention and are non-limiting. In accordance with the various examples, the evaluations and results demonstrate generally that the inventive compositions and methods confer beneficial results, including improved mechanical strength and quality of hair and improvement to the hydrophobicity of the hair.

Procedure for Preparation of Compositions for Application onto Hair

The compositions were generated by weighing the active RM(s) (raw material) into vials and stirring the RM(s) in the respective solvent. For example water was used as the solvent for the Carboxylic acid (Maleic acid) and for the Amine (MEA) and for the Polycarbodiimide; organic solvent such as isododecane was used for the Carboxylsilicone or a solvent mixture of water and organic solvent (e.g., isododecane) was used to make a composition containing maleic acid and MEA or CDI or both and Carboxysilicone. The vials were shaken vigorously by hand for 10 seconds/g of solution to create an aqueous solution.

TABLE 1
Representative Actives

| Generic name | Ingredient Name or Trade Name |
|---|---|
| Carboxylic acid 1 | Maleic acid or Cis-butenedioic acid, available |
| | from the supplier, Sigma Aldrich |
| Amine 1 | Ethanolamine or Monoethanolamine or 2- |
| | amino ethanol (“MEA”), available from the |
| | supplier, Aromat (BR) |
| Polycarbodiimide 1 | V-02-L2 (“CDI”) Nisshinbo |
| Carboxysilicone | Bis-Carboxydecyl dimethicone, trade name of |
| polymer 1 | SILFORM INX (supplied by Momentive |
| | Performance Materials) (“Si—COOH”) |

Protocols for Treating Hair

One-step, two-step and three-step application processes or methods were employed to treat hair.

Performance Assessments by Mechanical Property Tests Before and After Treatment of Hair With The Test Compositions in Reference to Untreated Hair.

General Protocol

The cross-sectional area of 50 fibers from each swatch was evaluated using a Laser Scanning Micrometer Fdas770 from Dia-Stron Limited. The values were determined by taking the average of 5 cross-sectional areas along 3 cm length of the fiber. Tensile properties were measured using a Dia-Stron Miniature Tensile Tester, MTT 686, extending the fibers at a rate of 15 mm/min. All tests were performed after the fibers equilibrated in the environmental chamber at 23±1° C. and 44±2% RH. Data was analyzed using the UV Win software.

Statistical Analysis: T

A one-way ANOVA analysis was performed using Tukey-Kramer statistical analysis to determine differences between controls and treated samples. Statistical significance is recognized at p<0.05.

Parameters Measured:

I. Plateau Stress Measurements—Plateau stress is a measure of the α-structure deformation of the fiber. Yield and plateau deformation are associated with molecular transition and measured in MPa. A significant increase of plateau stress values indicates that the fiber has a greater energy absorption capability before failure (breakage).

II. Break Stress Measurements—Break stress is a measure of the force required to break the fiber as measured in MPa. Significantly increased break stress values indicate reinforce hair cortex of bleach damaged hair.

III. Toughness Measurements—The toughness parameter is a measure of how strong and resistant the fiber is to breakage and is measured in J/m3.

An increase in toughness of the fiber indicates a reinforced cortex of the damaged bleached hair. Cycles to Break Measurements—Cyclic fatigue parameter is a measure of fiber durability (correlated to day to day grooming practices). Fiber durability is measured in number of cycles required to break the fiber using a constant stress value. A significant increase in durability or improvement in Durability (Characteristic lifetime) of damaged hair (such as bleached hair) is assessed in relation to the survival probability of natural hair. Durability/Characteristic lifetime=number cycles required to break 63.2% of the fibers

### Example I Treatment of Hair According to Protocol A

TABLE 2
Compositions

| | Composition A | Composition B | Composition C |
|---|---|---|---|
| Composition | (% by weight) | (% by weight) | (% by weight) |
| MA—MEA salt | 0.9 | — | 0.9 |
| complex* | | | |
| Polycarbodiimide 1 | — | 1 | 1 |
| Solvent (Water or | q.s to 100 | q.s to 100 | q.s to 100 |
| Isododecane or | | | |
| mixture of water | | | |
| and isododecane | | | |

*A pre-formed maleic acid-MEA salt complex contained Maleic acid, MA and MEA in a weight ratio of 2:1 of in water and colorants; the final active weights in the compositions above are: MA = 0.96% and MEA = 0.486%

Protocol A—One step application method. The following protocol was followed:

1) One gram bleached hair swatches (highly bleached hair) were washed once with a conventional shampoo in a 1:0.4 hair to shampoo weight ratio by wetting the hair with water for 5 seconds, then treating the hair with shampoo for 30 seconds, and then rinsing the hair with water for 30 seconds. After the hair was washed, the hair was wiped with a paper towel to remove excess water from the hair. The hair was damp but no water dripped. 0.4 gm of each test composition was applied to the hair using a plastic pipette, and the hair was combed well so that the test composition was evenly distributed.
2) The treated hair swatches were placed in a 50′C oven for 30 min.
3) The hair swatches were removed from the oven and placed in a 23±1° C. and 45±1% relative humidity (RH) environmental chamber for 48 hours.
4) Then the hair swatches were rinsed with warm water to remove excess solution and washed with the conventional shampoo once, then rinsed with water.
5) The hair swatches were blot dried with paper towel and dried with a blow drier set at about 70° C. for a minute.
6) After drying the hair swatches, they were placed in 23±1° C. and 4511% RH environmental chamber for at least 8 hours prior to assessing the hair quality (by mechanical and cyclic fatigue testing).

TABLE 3*
Plateau Stress Measurements (I)

| | | | Polycarbodiimide 1 | Composition C |
|---|---|---|---|---|
| Plateau stress (MPa) | No treatment | MA—MEA | (CDI) | (MA—MEA—CDI) |
| mean ± std. dev. | 121.2 ± 13.7 | 132.9 ± 17.5 | 133.7 ± 25.8 | 149.1 ± 25.8 |
| p-value**- Untreated | — | .029 | .017 | .000 |
| (SA45 hair) vs. Others | | | | |
| p- value- MA—MEA | .029 | — | .998 | .003 |
| vs. Others | | | | |
| p- value- CDI vs. Others | .017 | .998 | — | .005 |
| p-value- MA—MEA—CDI | .000 | .003 | .005 | — |
| vs. Others | | | | |

*Multiple Comparisons Table- Tukey HSD (Use SPSS Software)
**a p-value of less than 0.05 indicates that the difference is statistically significant.

Summary of Findings:

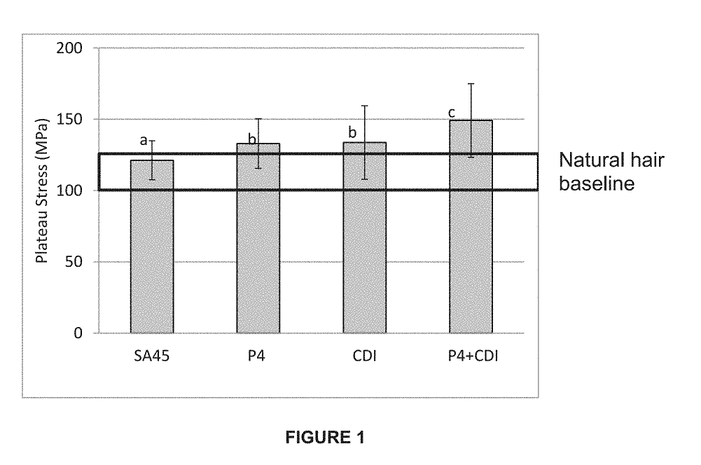

It was observed that the treatment of the hair with the composition C resulted in a statistically significant higher plateau stress value as compared to the values obtained from the treatment of the hair with the untreated and other compositions. See also, FIG. 1.

While the treatment with composition B (polycarbodiimide alone) resulted in a statistical difference when compared to no treatment on the hair, the mean plateau stress value was still significantly lower than the mean value obtained from the treatment with Composition C.

A significant increase of plateau stress value indicates that the hair fibers have a greater energy absorption capability. Therefore, the results indicate that the treatment of the hair with composition C, which contained the combination of carboxylic acid, amine and polycarbodiimide, imparted greater mechanical strength to bleach damaged hair.

TABLE 4*
Break Stress Measurements (II)

| | | | Polycarbodiimide 1 | Composition C |
|---|---|---|---|---|
| Break stress (MPa) | No treatment | MA—MEA | (CDI) | (MA—CDI) |
| mean ± std. dev. | 174.0 ± 36.1 | 187.9 ± 35.4 | 190.1 ± 44.2 | 238.3 ± 50.7 |
| p-value**- Untreated | — | .335 | .210 | .000 |
| (SA45 hair) vs. Others | | | | |
| p- value- MA—MEA | .335 | — | .993 | .000 |
| vs. Others | | | | |
| p- value- CDI vs. Others | .210 | .993 | — | .000 |
| p-value- MA—MEA—CDI | .000 | .000 | .000 | — |
| vs. Others | | | | |

*Multiple Comparisons Table- Tukey HSD (Use SPSS Software)
**a p-value of less than 0.05 indicates that the difference is statistically significant.

Summary of Findings:

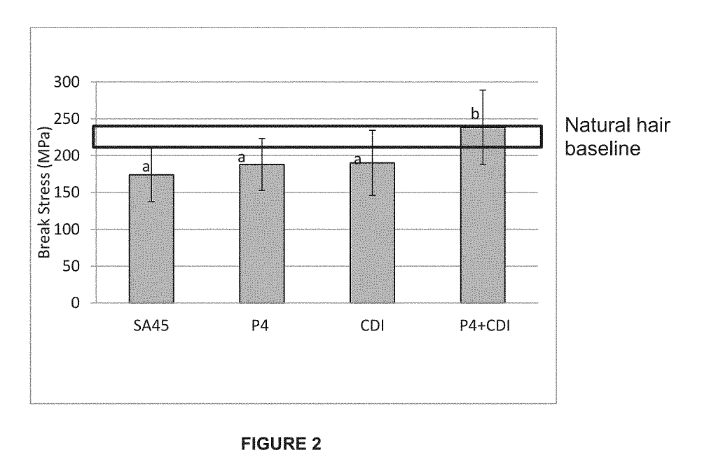

It was observed that the treatment of the hair with the composition C resulted in a statistically significant higher break stress value as compared to the values obtained from the treatment of the hair with the untreated and other compositions. See also, FIG. 2.

A significant increase of break stress value indicates that the hair fibers have a reinforced or stronger cortex. Therefore, the results indicate that the treatment of the hair with composition C imparted greater mechanical strength to bleach damaged hair.

TABLE 5*
Toughness Measurements

| | | | Polycarbodiimide 1 | Composition C |
|---|---|---|---|---|
| Toughness (107 J/m3) | No treatment | MA—MEA | (CDI) | (MA—CDI) |
| mean ± std. dev. | 5.54 ± 2.1 | 6.08 ± 2.1 | 6.12 ± 2.3 | 8.01 ± 1.9 |
| p-value**- Untreated | — | .589 | .519 | .000 |
| (SA45 hair) vs. Others | | | | |
| p- value- MA—MEA | .589 | — | 1.000 | .000 |
| vs. Others | | | | |
| p- value- CDI vs. Others | .519 | 1.000 | — | .000 |
| p-value- MA—MEA—CDI | .000 | .000 | .000 | — |
| vs. Others | | | | |

*Multiple Comparisons Table- Tukey HSD (Use SPSS Software)
**a p-value of less than 0.05 indicates that the difference is statistically significant.

Summary of Findings:

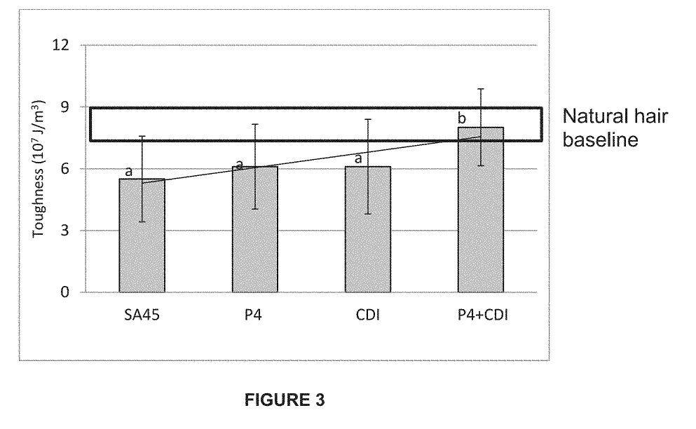

It was observed that the treatment of the hair with composition C resulted in a statistically significantly higher toughness value as compared to the values obtained from the treatment of the hair with the other compositions. It was also observed that the toughness values obtained from the treatment with the MA-MEA complex or polycarbodiimide 1 alone (compositions A and B) were not statistically significantly different from the toughness value obtained with the untreated fibers. See also, FIG. 3.

A significant increase of toughness value indicates that the hair fibers have a reinforced or stronger cortex. Therefore, the results indicate that the treatment of the hair with composition C imparted greater mechanical strength to bleach damaged hair.

### Example II Treatment of Hair According to Protocol B

TABLE 6
Compositions and Methods

| | | Test Methods and Compositions | | | | |
|---|---|---|---|---|---|---|
| | | | | | | 5 |
| | | | 2 | | | MA-MEA- |
| | | 1 | CDI, | 3 | 4 | CDI**, |
| | | MA-MEA, | Step 1 | MA-MEA- | CDI-Si—COOH, | step 1 |
| | | Step 1 | only | CDI Step | Step 2 | CDI-Si—COOH***, |
| | | only | (% by | 1 only | only | Step 2 |
| Protocol B | Ingredients | (% by wt) | wt) | (% by wt) | (% by wt) | (% by wt) |
| Step 1 | MA-MEA salt | 0.9 | — | 0.9 | — | 0.9 |
| | complex* | | | | | |
| | CDI | — | 1 | 1 | — | 1 |
| | Water | q.s to 100 | q.s to | q.s to | q.s to 100 | q.s to 100 |
| | | | 100 | 100 | | |
| Step 2 | CDI | | | | 0.33 | 0.33 |
| | Si—COOH | — | — | — | 1.667 | 1.667 |
| | Solvent | q.s to 100 | q.s to | q.s to | q.s to 100 | q.s to 100 |
| | (Water or | | 100 | 100 | | |
| | Isododecane | | | | | |
| | or mixture of | | | | | |
| | water and | | | | | |
| | isododecane | | | | | |

*A pre-formed maleic acid-MEA salt complex contained Maleic acid, MA and MEA in a weight ratio of 2:1 of in water and colorants; the final active weights in the compositions above are: MA = 0.96% and MEA = 0.486%.
**MA-MEA salt complex was mixed with CDI in a 1:1 weight ratio.
***CDI was mixed with Si—COOH for a net weight equivalent of 2% in the total weight of the CDI-Si—COOH composition.

Protocol B—Two step application method. The following protocol was followed:

1) One gram bleached hair swatches (SA45 type) were washed once with a conventional shampoo in a 1:0.4 hair to shampoo weight ratio by wetting the hair with water for 5 seconds, then treating the hair with shampoo for 30 seconds, and then rinsing the hair with water for 30 seconds.
2) After the hair was washed, the hair was wiped with a paper towel to remove excess water from the hair. The hair was damp but no water dripped. As a step 1 of the two-step application process, 0.4 gm of a test composition was applied to the hair using a plastic pipette, and the hair was combed well so that the test composition was evenly distributed.
3) The hair swatches were placed in a 50° C. oven for 30 min.
4) The hair swatches were removed from the oven and as a step 2 of the two-step application method, 0.4 gm of a test composition or ingredient was applied to the hair using a plastic pipette and then combed for a minute to evenly distribute.
5) The hair swatches were removed from the oven and placed in a 23±1° C. and 45±1% relative humidity (RH) environmental chamber for 48 hours.
6) Then the hair swatches were rinsed with warm water to remove excess solution and washed with the conventional shampoo once, then rinsed with water.
7) The hair swatches were blot dried with paper towel and dried with a blow drier at a setting of about 70° C. for a minute.
8) After drying the hair swatches, they were placed in 23±1° C. and 45±11% RH environmental chamber for at least 8 hours prior to assessing the hair quality (by mechanical testing).

TABLE 7*
Mechanical Testing - Cycles to Break Measurements (IV)

| | | Test Methods and Compositions | | | | |
|---|---|---|---|---|---|---|
| | | | | 3 | | |
| | | 1 | | MA- | | 5 |
| | | MA- | 2 | MEA- | 4 | MA-MEA- |
| | | MEA, | CDI, | CDI, | CDI-Si—COOH, | CDI, step 1 |
| | | Step 1 | Step 1 | Step 1 | Step 2 | CDI-Si—COOH, |
| Cycles to Break | Untreated | only | only | only | only | Step 2 |
| mean ± std. dev. | 3781.2 ± 948.1 | 4142.3 ± 899.9 | 3760.0 ± 659.0 | 3579 ± 884.3 | 2598.0 ± 701.8 | 11252.5 ± 1593.5 |
| p-value** - | — | .785 | .793 | .997 | .206 | .000 |
| Untreated (SA45 | | | | | | |
| hair) vs. Others | | | | | | |
| p-value - Test | .785 | — | .418 | .868 | .086 | .000 |
| Method 1 vs. | | | | | | |
| Others | | | | | | |
| p-value - Test | .793 | .418 | — | .904 | .428 | .000 |
| Method 2 vs. | | | | | | |
| Others | | | | | | |
| p-value - Test | .997 | .868 | .904 | | .915 | .000 |
| Method 3 vs. | | | | | | |
| Others | | | | | | |
| p-value - Test | .206 | .086 | .426 | .000 | — | .000 |
| Method 4 vs. | | | | | | |
| Others | | | | | | |
| p-value - Test | .000 | .000 | .000 | 0.000 | .000 | — |
| Method 5 vs. | | | | | | |
| Others | | | | | | |

*Multiple Comparisons Table - Tukey HSD (Use SPSS Software)
**a p-value of less than 0.05 indicates that the difference is statistically significant.

Summary of Findings:

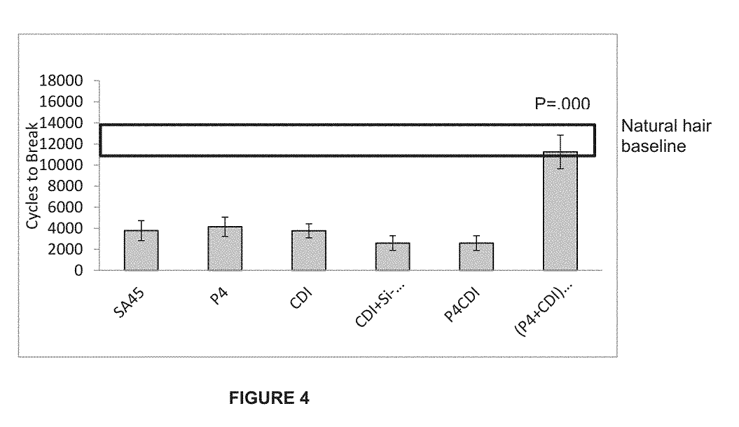

It was observed that the treatment of the hair according to the two-step application method (5) of the invention resulted in a statistically significant higher number of cycles to break value as compared to the values obtained from the treatment of the hair according to the other test methods. Moreover, the high number of cycles to break value obtained by using the method of the invention is close to that of natural hair. In addition, no significant differences were found among the other test methods (1 to 4 and untreated). See also, FIG. 4.

A significantly higher number of cycles to break value indicates that the hair fibers are more durable and therefore, have a longer lifetime. Therefore, the results indicate that the treatment of the hair according to the method of the invention imparted greater mechanical strength to bleach damaged hair.

### Example III Treatment of Hair According to Protocol C

TABLE 8
Compositions and Methods

| | | Test Methods and Compositions | | |
|---|---|---|---|---|
| | | | | 8 |
| | | 6 | 7 | MA—MEA, step 1 |
| | | MA—MEA, | CDI, | CDI, Step 2 |
| | | Step 1 only | Step 2 only | Si—COOH, Step 3 |
| Protocol C | Ingredients | (% by wt) | (% by wt) | (% by wt) |
| Step 1 | MA—MEA salt complex* | 0.9 | — | 0.9 |
| Step 2 | CDI | — | 1 | 1 |
| Step 3 | Si—COOH | — | — | 1 |
| | Solvent (Water or | q.s to 100 | q.s to 100 | q.s to 100 |
| | Isododecane or mixture | | | |
| | of water and isododecane | | | |

*A pre-formed maleic acid-MEA salt complex contained Maleic acid, MA and MEA in a weight ratio of 2:1 of in water and colorants; the final active weights in the compositions above are: MA = 0.96% and MEA = 0.486%.

Protocol C—Three step application method. The following protocol was followed:

1) One gram bleached hair swatches (SA45 type) were washed once with a conventional shampoo in a 1:0.4 hair to shampoo weight ratio by wetting the hair with water for 5 seconds, then treating the hair with shampoo for 30 seconds, and then rinsing the hair with water for 30 seconds. After the hair was washed, the hair was wiped with a paper towel to remove excess water from the hair. The hair was damp but no water dripped. As a step 1 of the three-step application method, 0.4 gm of a test composition was applied to the hair using a plastic pipette, and the hair was combed well so that the test composition was evenly distributed.
2) The hair swatches were allowed to sit for 30 min at room temperature.
3) As a step 2 of the three-step application method, 0.2 gm of a test composition was applied to the hair using a plastic pipette and the hair was gently combed for 1 minute for even distribution.
4) As a step 3 of the three-step application method, 0.2 gm of a test composition was applied to the hair using a plastic pipette and the hair was gently combed for 1 minute for even distribution.
5) The hair swatches were placed in a 50° C. oven for 30 min.
6) The hair swatches were removed from the oven and placed in a 23±1° C. and 45±1% relative humidity (RH) environmental chamber for 48 hours.
7) Then the hair swatches were rinsed with warm water to remove excess solution and washed with the conventional shampoo once, then rinsed.
8) The hair swatches were blot dried with paper towel and dried with a blow drier set at about 70° C. for a minute.
9) After drying the hair swatches, they were placed in 23±1° C. and 45±1% RH environmental chamber for at least 8 hours prior to assessing the hair quality (by mechanical testing).

TABLE 9*
Plateau Stress Measurements (I)

| | | Test Methods and Compositions | | | |
|---|---|---|---|---|---|
| | | | | | 8 |
| | | 6 | 7 | 9 | MA—MEA, Step 1 |
| | | MA—MEA, | CDI, | CDI—Si—COOH***, | CDI, Step 2 |
| Plateau stress (MPa) | No treatment | Step 1 only | Step 2 only | Step 3 only | Si—COOH, Step 3 |
| mean ± std. dev. | 121.21 ± 13.7 | 132.94 ± 17.5 | 133.68 ± 25.8 | 125.1 ± 20.4 | 160.77 ± 24.7 |
| p-value**- Untreated | — | .043 | .026 | .920 | .000 |
| (SA45 hair) vs. Others | | | | | |
| p- value- Test Method 9 | .043 | — | −1 | .429 | .000 |
| vs. Others | | | | | |
| p- value- Test Method 10 | .026 | 1 | — | .337 | .000 |
| vs. Others | | | | | |
| p- value- Test Method 11 | .920 | .429 | .337 | — | .000 |
| vs. Others | | | | | |
| p-value- Test Method 12 | .000 | .000 | .000 | .000 | — |
| vs. Others | | | | | |

*Multiple Comparisons Table- Tukey HSD (Use SPSS Software)
**a p-value of less than 0.05 indicates that the difference is statistically significant
***CDI was mixed with Si—COOH for a net weight equivalent of 2% in the total weight of the CDI—Si—COOH composition

Summary of Findings:

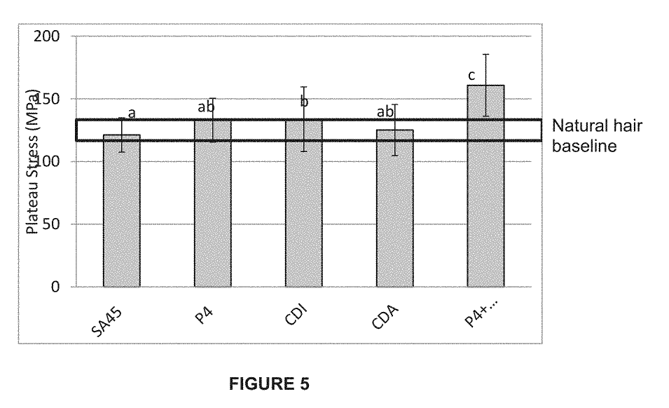

It was observed that the treatment of the hair according to the three-step application method (8) of the invention resulted in a statistically significant higher plateau stress value as compared to the values obtained from the treatment of the hair according to the other test methods. See also, FIG. 5.

A significant increase of plateau stress value indicates that the hair fibers have a greater energy absorption capability. Therefore, the results indicate that the treatment of the hair according to the method of the invention imparted greater mechanical strength to bleach damaged hair.

TABLE 10*
Break Stress Measurements (II)

| | | Test Method | | | |
|---|---|---|---|---|---|
| | | | | | 8 |
| | | 6 | 7 | 9*** | MA—MEA, Step 1 |
| | | MA—MEA, | CDI, | CDI—Si—COOH, | CDI, Step 2 |
| Break stress (MPa) | No treatment | Step 1 only | Step 2 only | Step 3 only | Si—COOH, Step 3 |
| mean ± std. dev. | 174.0 ± 36.1 | 187.9 ± 35.4 | 190.1 ± 44.2 | 179.3 ± 40.4 | 239.7 ± 43.8 |
| p-value**- Untreated | — | .416 | .265 | .976 | .000 |
| (SA45 hair) vs. Others | | | | | |
| p- value- Test Method 9 | .416 | — | .999 | .865 | .000 |
| vs. Others | | | | | |
| p- value- Test Method 10 | .265 | .265 | — | .735 | .000 |
| vs. Others | | | | | |
| p- value- Test Method 11 | .976 | .976 | .736 | — | .000 |
| vs. Others | | | | | |
| p-value- Test Method 12 | .000 | .000 | .000 | .000 | — |
| vs. Others | | | | | |

*Multiple Comparisons Table- Tukey HSD (Use SPSS Software)
**a p-value of less than 0.05 indicates that the difference is statistically significant.
***CDI was mixed with Si—COOH for a net weight equivalent of 2% in the total weight of the CDI—Si—COOH composition.

Summary of Findings:

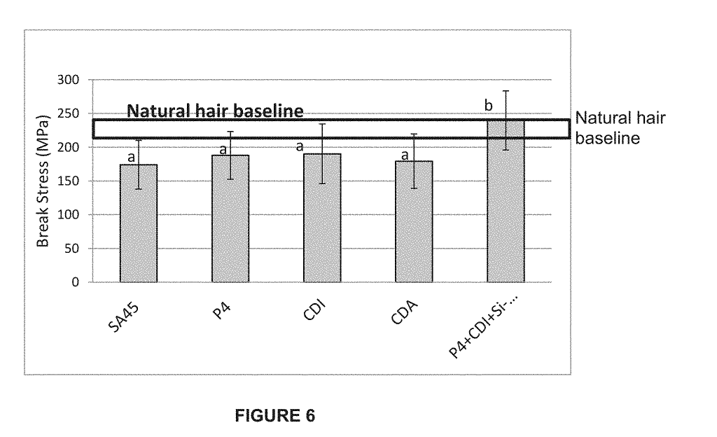

It was observed that the treatment of the hair according to the three-step application method (8) of the invention resulted in a statistically significant higher break stress value as compared to the values obtained from the treatment of the hair according to the other test methods. In addition, no significant differences were found among the other test methods (6, 7, 9 and untreated). See also, FIG. 6.

A significant increase of break stress value indicates that the hair fibers have a reinforced or stronger cortex. Therefore, the results indicate that the treatment of the hair according to the method of the invention imparted greater mechanical strength to hair, i.e., it reinforced the cortex of bleach damaged hair.

TABLE 11*
Toughness Measurements

| | | Test Method | | | |
|---|---|---|---|---|---|
| | | | | | 8 |
| | | 6 | 7 | 9** | MA—MEA, Step 1 |
| | | MA—MEA, | CDI, | CDI—Si—COOH, | CDI, Step 2 |
| Toughness (107 J/g) | No treatment | Step 1 only | Step 2 only | Step 3 only | Si—COOH, Step 3 |
| mean ± std. dev. | 5.55 ± 2.1 | 6.08 ± 2.1 | 6.12 ± 2.3 | 6.37 ± 2.0 | 7.88 ± 2.1 |
| p-value**- Untreated | — | .721 | .654 | .392 | .000 |
| (SA45 hair) vs. Others | | | | | |
| p- value- Test Method 9 | .721 | — | 1 | .969 | .000 |
| vs. Others | | | | | |
| p- value- Test Method 10 | .654 | 1 | — | .983 | .000 |
| vs. Others | | | | | |
| p- value- Test Method 11 | .392 | .969 | .983 | — | .013 |
| vs. Others | | | | | |
| p-value- Test Method 12 | .000 | .000 | .000 | .013 | — |
| vs. Others | | | | | |

*Multiple Comparisons Table- Tukey HSD (Use SPSS Software)
**a p-value of less than 0.05 indicates that the difference is statistically significant.
***CDI was mixed with Si—COOH for a net weight equivalent of 2% in the total weight of the CDI—Si—COOH composition

Summary of Findings:

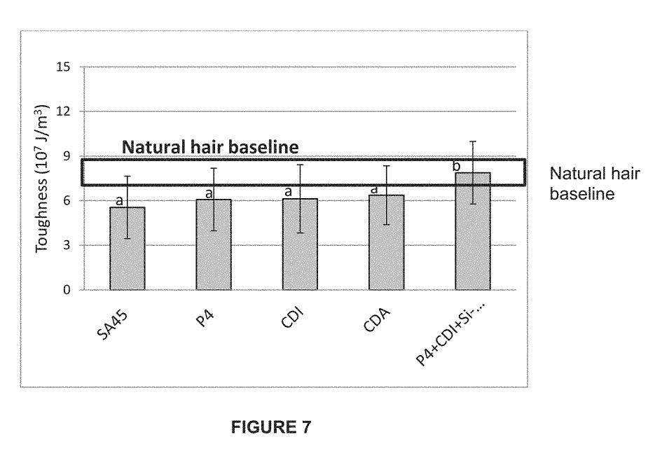

It was observed that the treatment of the hair according to the three-step application method of the invention resulted in a statistically significantly higher toughness value as compared to the values obtained from the treatment of the hair according to the other test methods. It was also observed that the toughness values obtained from the treatment with the other non-inventive test methods were not statistically significantly different from the toughness value obtained with the untreated fibers. See also, FIG. 7.

A significant increase of toughness value indicates that the hair fibers have a reinforced or stronger cortex. Therefore, the results indicate that the treatment of the hair according to the three-step application method of the invention imparted greater mechanical strength to bleach damaged hair.

TABLE 12*
Mechanical Testing - Cycles to Break Measurements (IV)

| | | Test Method | | | |
|---|---|---|---|---|---|
| | | | | | 8 |
| | | 6 | 7 | 9*** | MA—MEA, Step 1 |
| | | MA—MEA, | CDI, | CDI—Si—COOH, | CDI, Step 2 |
| Cycles to Break | No treatment | Step 1 only | Step 2 only | Step 3 only | Si—COOH, Step 3 |
| mean ± std. dev. | 3583.1 ± 747.9 | 4142.3 ± 899.9 | 3760.0 ± 659.0 | 1398.1 ± 295.4 | 12352.5 ± 1423.7 |
| p-value**- Untreated | — | .439 | .965 | .017 | .000 |
| (SA45 hair) vs. Others | | | | | |
| p- value- Test Method 9 | .439 | — | .418 | .013 | .000 |
| vs. Others | | | | | |
| p- value- Test Method 10 | .965 | .418 | — | .002 | .000 |
| vs. Others | | | | | |
| p- value- Test Method 11 | .000 | .000 | .000 | — | .000 |
| vs. Others | | | | | |
| p-value- Test Method 12 | .000 | .000 | .000 | .000 | — |
| vs. Others | | | | | |

*Multiple Comparisons Table- Tukey HSD (Use SPSS Software)
**a p-value of less than 0.05 indicates that the difference is statistically significant.
***CDI was mixed with Si—COOH for a net weight equivalent of 2% in the total weight of the CDI—Si—COOH composition.

Summary of Findings:

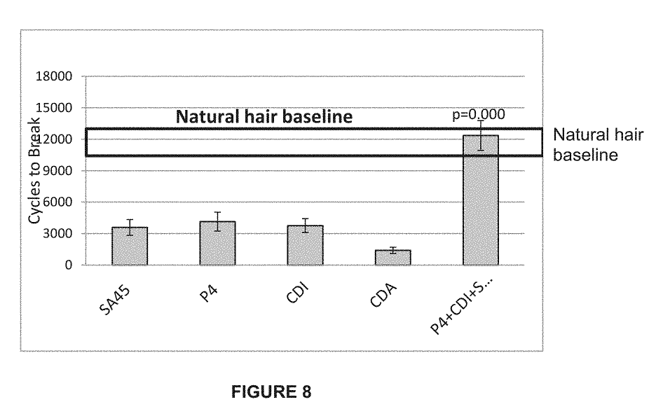

It was observed that the treatment of the hair according to the three-step application method (8) of the invention resulted in a statistically significant higher number of cycles to break value as compared to the values obtained from the treatment of the hair according to the other test methods. In addition, the cycles to break values obtained using the other test methods (6, 7, 9) and for the untreated hair were very low. See also, FIG. 8.

A significantly higher number of cycles to break value indicates that the hair fibers are more durable and therefore, have a longer lifetime. Therefore, the results indicate that the treatment of the hair according to the method of the invention imparted greater mechanical strength to bleach damaged hair.

### Example IV Treating Hair with Compositions Containing Active Agents and Evaluating the Surface of Hair

The condition of the surface of hair was evaluated in terms of its hydrophobic properties.

Hydrophobicity of hair (swatches) was measured via contact angle measurements using Biolin Scientific Contact Angle Tensiometer, Model C204A. A bundle of 30-50 fibers was clamped to create a flat surface. A 3 μL drop of DI (deionized) H2O was placed on the fiber surface and the contact angle was measured for 10 seconds. The values reported below are an average of 3 measurements using the contact angle at 10 seconds. Natural, undamaged hair is hydrophobic while damaged hair (double bleached, or platinum bleached) is hydrophilic and exhibits a contact angle of 0.°

The cationic nature of the surface was evaluated by a red-80 dye test. A stock solution of Red 80 was made with the following formula:

| Raw Material | Wt % |
|---|---|
| Red 80 | 0.5 |
| Acetic Acid | 0.13 |
| DI Water | 99.37 |

10.8 g of the stock solution was diluted to 54 g with deionized water. The swatch was soaked for 5 minutes in this solution. Excess dye was removed by soaking the swatch for 1 minute in 100 mL of deionized water, which was then repeated with clean deionized water 4 additional times. The swatch was then dried.

Each swatch tested required its own solution of Red 80 dye as well as 5 beakers of fresh deionized water for rinsing. As the Red 80 dye is anionic, a cationic surface (hair) will attract the dye, thereby resulting in a red swatch. The more intense the red, after rinsing and drying, the more cationic charges are on the surface of the hair.

Controls: Treatment of Hair with Individual Components

Test application methods: Hair was treated according to two different methods—a two-step chemical treatment application method or a three-step chemical treatment application method. The term “chemical treatment” as used herein refers to a composition that contains one or more Active Agents as defined and used in the present disclosure. It additionally contains the MA-MEA salt complex. In the examples below, a chemical treatment composition was prepared by mixing a developer composition containing oxidizing agents and a bleach composition containing bleaching agents.

Step 1 of both methods involved treating hair with a chemical treatment composition containing an Active Agent and the MA-MEA salt complex

In some of the test application methods, a rinse-off conditioning composition (conditioner) was used as an alternative step in the two-step chemical treatment application method or as an additional step in the three-step chemical treatment application method.

Compositions

Developer Formulations with Oxidizing Agents

| Component | 30V Developer | 40V Developer |
|---|---|---|
| Hydrogen Peroxide | 18 | 24 |
| (oxidizing agent) | | |
| Pentasodium Pentetate | 0.15 | 0.15 |
| Surfactants | 3.7 | 3.7 |
| Organic solvent | 0.5 | 0.5 |
| Additives | 0.06 | 0.06 |
| Water | QS 100 | QS 100 |

Bleach Powder Composition with Bleaching Agents

| Ingredient | wt. % |
|---|---|
| Magnesium Carbonate Hydroxide | 9 |
| Sodium Silicate and Sodium Metasilicate | 20 |
| (bleaching agents) | |
| Disodium EDTA | 1 |
| Surfactants | 11 |
| Potassium Persulfate (bleaching agents) | 50 |
| Ammonium Persulfate (bleaching agents) | 5 |
| Additives and Optional Ingredients | 4 |

15 g of the bleach powder and 15 g of the bleach developer (either 30 or 40V) were mixed with 4 g of the maleic acid-MEA salt complex (2:1 weight ratio in water and colorants) to form a chemical treatment composition for hair. The final active weights of MA and MEA in the chemical treatment composition was MA=2.3 wt % and MEA=1.1 wt %

CDI-SiCOOH (Mixture of CDI & Si—COOH)

| | | % by weight |
|---|---|---|
| Phase | Raw Material | (Active) |
| A | Water | 88.2 |
| A | hydroxyethylcellulose | 0.9 |
| A | Phenoxyethanol | 0.8 |
| A | Ethylhexyl glycerine | 0.1 |
| A | CDI (V02-L2) | 0.9 |
| B | Isododecane | 8.2 |
| B | Carboxysilicone polymer 1 | 0.9 |

Rinse-Off Conditioning Composition (Conditioner)

| | INGREDIENTS* | % by weight |
|---|---|---|
| | INCI US NAMES | (active) |
| Cationic | CETRIMONIUM CHLORIDE, | 1.9 |
| conditioning | CETRIMONIUM METHOSULFATE, | |
| agents | BEHENTRIMONIUM | |
| | METHOSULFATE, | |
| | STEARAMIDOPROPYL | |
| | DIMETHYLAMINE | |
| Fatty compounds | CETEARYL ALCOHOL, MINERAL | 4.3 |
| | OIL | |
| Film formers | ACRYLATES COPOLYMER, | 0.212 |
| | POLYQUATERNIUM-37 | |
| Thickening | GLYCERIN, PROPYLENE GLYCOL, | 4.0 |
| agents, polyols | HYDROXYETHYLCELLULOSE | |
| Other ingredients | PRESERVATIVES, NONIONIC | 0.72 |
| | SURFACTANTS | |
| solvent | WATER | QS 100 |

*composition can also contain the MA—MEA salt complex

Protocol D: The two-step chemical treatment application method is a follows:

1) Commercial bleached hair swatches (regular bleached hair) were bleached for 35 minutes using a 1:1:0.27 mixture of the bleach powder (15 g), the 40V developer (15 g), and the maleic acid-monoethanolamine salt complex solution (4 g), and the hair swatches were rinsed with water.
2) A leave-on treatment composition (CDI—Si—COOH) containing Polycarbodiimide 1 (CDI) and Carboxysilicone 1 polymer (Si—COOH) in a weight ratio of 1:1 was applied onto the swatches.

For a comparative test, instead of step 2 above, the hair swatches were treated with the rinse-off conditioning composition (conditioner described above). The conditioner was allowed to remain for about 10 minutes, after which it was rinsed off from the hair with water. The hair swatches treated according to protocol D (invention) and to the comparative test were assessed using sensorial evaluation for cosmeticity and contact angle measurements for hydrophobicity.

TABLE 13
Sensorial Evaluation

| | | Step 2′ | # of shampoos | | |
|---|---|---|---|---|---|
| Step 1 | Step 2 | (comparative) | after treatment | Combability* | Discipline* |
| —** | — | — | 1x | 3 | 3 |
| MA—MEA | — | Conditioner | 1x | 2 | 2 |
| MA—MEA | CDI—Si—COOH | — | 1x | 1 | 1 |

*ratings by a human evaluator; the lower the rating, the better the sensorial attributes of combability (ease of combing, less tangling) and discipline (less flyaways, less frizzy)
**No treatment or untreated (refers to treatment only with bleach-developer mixture)

A commercial, clarifying shampoo was used on the hair after treating the hair with the compositions of the invention

The results in the above table shows that the method of the invention comprising steps 1 and 2 yielded the best sensorial ratings over the comparative method comprising steps 1 and 2′ and over no treatment.

TABLE 14
Contact Angle Measurements

| | | | # of | |
|---|---|---|---|---|
| | | Step 2′ | shampoos | |
| | | (compar- | after | Contact |
| Step 1 | Step 2 | ative) | treatment | Angle |
| —** | — | — | 1x | 0° |
| —** | — | — | 6x | 0° |
| MA—MEA | — | Conditioner | 1x | 0° |
| MA—MEA | — | Conditioner | 6x | 0° |
| MA—MEA | CDI—Si—COOH | — | 1x | 121° ± 3 |
| MA—MEA | CDI—Si—COOH | — | 6x | 119° ± 2 |

Swatches were evaluated over 6 shampoo cycles (shampoo/rinse) with a commercial shampoo.

The contact angle measurements demonstrate that only the swatches treated according to the inventive method comprising Steps 1 and 2 were hydrophobic and this effect is retained over the 6 shampoo cycles. It is evident that the quality of the hair that has been damaged by a bleach-developer treatment has been restored or improved to a condition or state that is close to or similar to the condition of undamaged hair which is hydrophobic. This benefit conferred to the hair was found to be long lasting or durable over multiple washings or shampoo cycles.

Protocol E: The three-step chemical treatment application method is as follows:

1) Commercial bleached hair swatches (regular bleached hair) were bleached for 35 minutes using a mixture of the bleach powder, the 40V developer, and the maleic acid-monoethanolamine mixture (MA-MEA salt complex mixture) and the hair swatches were rinsed with water.
2) The rinse-off conditioning composition (conditioner) was applied onto the hair and allowed to remain for about 10 minutes, after which it was rinsed off from the hair with water.
3) A leave-on treatment composition (CDI—Si—COOH) containing Polycarbodiimide 1 (CDI) and Carboxysilicone 1 polymer (Si—COOH) in a weight ratio of 1:1 was applied onto the swatches.

For a comparative test, the hair swatches were not subjected to step 3 (no treatment with CDI—Si—COOH) The hair swatches treated according to protocol E (invention) and to the comparative test were assessed using sensorial evaluation for cosmeticity and contact angle measurements for hydrophobicity.

TABLE 15
Sensorial Evaluation

| | | | # of | | |
|---|---|---|---|---|---|
| | | | shampoos | | |
| | | Step 3 | after | Comb- | Disci- |
| Step 1 | Step 2 | (comparative) | treatment | ability* | pline* |
| —** | — | — | 1x | 3 | 3 |
| MA—MEA | Condi- | — | 1x | 2 | 2 |
| | tioner | | | | |
| MA—MEA | Condi- | CDI—Si—COOH | 1x | 1 | 1 |
| | tioner | | | | |

*ratings by a human evaluator; the lower the rating, the better the sensorial attributes of combability (ease of combing, less tangling) and discipline (less flyaways, less frizzy)
**No treatment or untreated (refers to treatment only with bleach-developer mixture)

A commercial shampoo was used on the hair after treating the hair with the compositions of the invention

The results in the above table shows that the method of the invention comprising steps 1, 2 and 3 yielded the best sensorial ratings over the comparative method comprising steps 1 and 2 only and over no treatment.

TABLE 16
Red 80 Dye Evaluation and Contact Angle Measurements

| | | | # of | | | | |
|---|---|---|---|---|---|---|---|
| | | | shampoos | | | | |
| | | | after | | | | Contact |
| Step 1 | Step 2 | Step 3 | treatment | L | A | B | Angle |
| —** | — | — | 1x | | | | 0° |
| —** | — | — | 6x | | | | 0° |
| MA- | Conditioner | — | 1x | 52.78 | 20.44 | 18.01 | 0° |
| MEA | | | | | | | |
| MA- | Conditioner | — | 6x | 53.82 | 19.14 | 18.50 | 0° |
| MEA | | | | | | | |
| MA- | Conditioner | CDI-Si—COOH | 1x | 50.98 | 23.01 | 18.35 | 124° ± 5 |
| MEA | | | | | | | |
| MA- | Conditioner | CDI-Si—COOH | 6x | 51.09 | 21.14 | 16.54 | 1190° ± 9 |
| MEA | | | | | | | |

Swatches were evaluated over 6 shampoo cycles with a commercial shampoo.

In summary, the smaller L value and larger A value show that the swatches treated with the three-step chemical treatment application method resulted in darker, redder swatches; this result was retained over 6 shampoo cycles. At the same time, only the swatches treated according to the three-step chemical treatment application method demonstrated hydrophobic properties which were retained over 6 shampoo cycles. It is evident from the contact angle results that the quality of the hair that has been damaged by a bleach-developer treatment has been restored or improved to a condition or state that is close to or similar to the condition of undamaged hair which is hydrophobic. This benefit conferred to the hair was found to be long lasting or durable over multiple washings or shampoo cycles. It is also evident that the CDI-SiCOOH treatment not only contributes to conferring hydrophobicity to the hair (same as protocol D) but also helps to keep longer lasting or more durable conditioning benefits over multiple shampoos as conferred by the conditioner on the fibers (compared to the conditioning benefits conferred when the CDI-SiCOOH composition was not used).
While the invention has been described with reference to certain exemplary or preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An agent for treating keratinous fibers comprising one or more separately contained treatment compositions, wherein each of the one or more treatment compositions include at least one of the following components:
(a) at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and mixtures thereof;
(b) at least one amine chosen from alkyl amines, alkanolamines, alkylene amines, and mixtures thereof;
(c) at least one polycarbodiimide compound; and
(d) at least one carboxysilicone polymer;
provided that components (a) and (c) are included in the one or more treatment compositions.

2. The agent for treating keratinous fibers according to claim 1, wherein the at least one amine is chosen from monoethanolamine (MEA), diethanolamine (DEA), triethanolamine (TEA), monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, 3-isopropoxypropylamine, 3-methoxypropylamine (3-MPA), tris(hydroxymethyl)aminomethane, 3-ethoxypropylamine, 3-(2-ethylhexyloxy)-propylamine, 2-(2-aminoethoxy)ethanol (2-2AEE), 3-butoxypropylamine (3-BPA), ethylamines, ethyleneamine, and mixtures thereof.

3. The agent for treating keratinous fibers according to claim 1, wherein the at least one polycarbodiimide compound has the following formula: 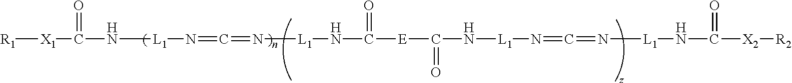
wherein X₁ and X₂ each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₆ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

4. The agent for treating keratinous fibers according to claim 1, wherein the at least one polycarbodiimide compound is a co-polymer derived from alpha-methylstyryl-isocyanates having the following formula: 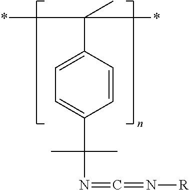
wherein R is an alkyl, cycloalkyl or aryl group having from 1 to 24 carbon atoms.

5. The agent for treating keratinous fibers according to claim 1, wherein the at least one polycarbodiimide compound is a compound having the following structure: 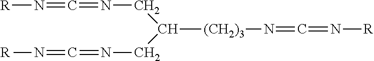
wherein R is an alkyl, cycloalkyl or aryl group.

6. The agent for treating keratinous fibers according to claim 5, wherein R is an alkyl, cycloalkyl or aryl group having from 1 to 24 carbon atoms.

7. The agent for treating keratinous fibers according to claim 1, wherein the at least one carboxysilicone polymer is a compound having at least one carboxylate group and is chosen from:
(A) a compound having the following formula: 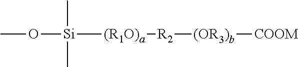
wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or
(B) a compound having a group comprising at least one pyrrolidone carboxylic acid unit having the following formula: 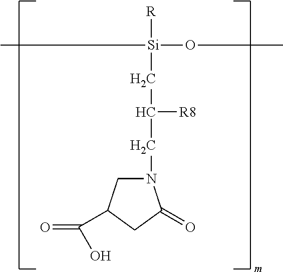
wherein R is selected from methyl or phenyl; R8 is hydrogen or methyl, m is an integer from 1 to 1000;
(C) a compound having a group comprising at least one polyvinyl acid/ester unit resulting from the polymerization of Divinyl-PDMS, Crotonic Acid, Vinylacetate, and Vinyl Isoalkylester;
and combinations thereof.

8. The agent for treating keratinous fibers according to claim 1, wherein the at least one carboxysilicone polymer is a compound having at least one carboxylate group and the formula: 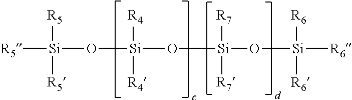
wherein the radicals R₄ are identical to or different from each other and are chosen from a linear or branched C₁-C₂₂ alkyl radical, a C₁-C₂₂ alkoxy radical and a phenyl radical, the radicals R₅, R₅′, R₅″, R₆, R₆′, R₆″, R₇, and R₇′ are identical to or different from each other and are chosen from a linear or branched C₁-C₂₂ alkyl radical, a C₁-C₂₂ alkoxy radical, a phenyl radical, a radical —(R₁O)ₐ—R₂—(OR₃)_{b}—COOM, a radical containing pyrrolidone carboxylic acid, a radical of polyvinyl acid/ester; and
wherein at least one of the radicals R₅, R₆ and R₇ is a radical chosen from a radical —(R₁O)ₐ—R₂—(OR₃)_{b}—COOM, a radical containing pyrrolidone carboxylic acid, a radical of polyvinyl acid/ester, a radical of polyvinyl acid/ester;
wherein R1 and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene;
wherein c and d are integers from 0 to 1000, the sum c+d ranging from 2 to 1000

9. The agent for treating keratinous fibers according to claim 1, wherein the one or more treatment compositions comprises a solvent chosen from water, organic solvents, and a mixture thereof.

10. The agent for treating keratinous fibers according to claim 1, wherein the agent for treating keratinous fibers includes amounts of each of the at least one polycarbodiimide compound, the at least one carboxylic acid, the at least one amine, and the at least one carboxysilicone polymer sufficient to impart to the keratinous fibers after application thereto, one or more of:
improved conditioning;
protection from damage caused by extrinsic and/or intrinsic factors;
repair when the keratinous fibers are in a damaged condition;
minimized or no breakage;
increased or improved mechanical strength;
increased hydrophobicity; or
increased hold to the shape or configuration of the fibers.

11. The agent for treating keratinous fibers according to claim 1, wherein at least one of the one or more treatment compositions is a treatment composition comprising:
the at least one carboxylic acid in an amount, by weight, of from about 0.02% to about 5%, based on the total weight of the composition; and
the at least one polycarbodiimide compound in an amount, by weight, of from about 0.25% to about 20%, based on the total weight of the composition.

12. The agent for treating keratinous fibers according to claim 1, wherein at least one of the one or more treatment compositions is a treatment composition comprising:
the at least one carboxylic acid in an amount, by weight, of from about 0.02% to about 5%, based on the total weight of the composition;
the at least one amine is present in an amount, by weight, of from about 0.02% to about 5%, based on the total weight of the composition; and
the at least one polycarbodiimide compound in an amount, by weight, of from about 0.25% to about 20%, based on the total weight of the composition.

13. A method of treating keratinous fibers, comprising applying onto keratinous fibers, the treatment composition of claim 12 and wherein the keratinous fibers include hair.

14. The agent for treating keratinous fibers according to claim 1, wherein at least one of the one or more treatment compositions is (1) a first treatment composition comprising:
the at least one carboxylic acid in an amount, by weight, of from about 0.02% to about 5%, based on the total weight of the first treatment composition;
the at least one amine is present in an amount, by weight, of from about 0.02% to about 5%, based on the total weight of the first treatment composition; and
the at least one polycarbodiimide compound in an amount, by weight, of from about 0.1% to about 10%, based on the total weight of the first treatment composition;
wherein at least one of the one or more treatment compositions is (2) a second treatment composition comprising:
the at least one polycarbodiimide compound in an amount, by weight, of from about 0.21% to about 10%, based on the total weight of the second treatment composition; and
the at least one carboxysilicone polymer, in an amount, by weight, of from about 0.25% to about 20%, based on the total weight of the second treatment composition.

15. The agent for treating keratinous fibers according to claim 13, wherein the weight ratio of the at least one carboxylic acid to the at least one amine in the first treatment composition ranges from 10:1 to 1:10.

16. A method of treating keratinous fibers, comprising:
(i) applying the first treatment composition of claim 14 onto the fibers;
(ii) heating the fibers at a temperature above room temperature; and
(III) applying the second treatment composition of claim 14 onto the fibers;
wherein the keratinous fibers include hair.

17. The agent for treating keratinous fibers according to claim 1,
wherein at least one of the one or more treatment compositions is (1) a first treatment composition comprising:
the at least one carboxylic acid in an amount, by weight, of from about 0.02% to about 5%, based on the total weight of the first treatment composition; and
the at least one amine is present in an amount, by weight, of from about 0.02% to about 5%, based on the total weight of the first treatment composition; and
wherein at least one of the one or more treatment compositions is (2) a second treatment composition comprising:
the at least one polycarbodiimide compound in an amount, by weight, of from about 0.1% to about 10%, based on the total weight of the second treatment composition; and
wherein at least one of the one or more treatment compositions is (3) a third treatment composition comprising:
the at least one carboxysilicone polymer, in an amount, by weight, of from about 0.25% to about 20%, based on the total weight of the third treatment composition.

18. A method of treating keratinous fibers, comprising:
(i) applying the first treatment composition of claim 17 onto the fibers;
(ii) leaving the first treatment composition on the fibers at room temperature for at least 30 minutes;
(III) applying the second treatment composition of claim 17 onto the fibers;
(iv) applying the third treatment composition of claim 17 onto the fibers; and
(v) heating the fibers at a temperature above room temperature;
wherein the keratinous fibers include hair.

19. The agent for treating keratinous fibers according to claim 1,
wherein at least one of the one or more treatment compositions is (1) a first treatment composition comprising:
the at least one carboxylic acid in an amount, by weight, of from about 0.02% to about 5%, based on the total weight of the first treatment composition;
the at least one amine is present in an amount, by weight, of from about 0.02% to about 5%, based on the total weight of the first treatment composition; and
at least one hair active agent;
wherein at least one of the one or more treatment compositions is (2) a second treatment composition comprising:
the at least one polycarbodiimide compound in an amount, by weight, of from about 0.1% to about 10%, based on the total weight of the second treatment composition; and
the at least one carboxysilicone polymer, in an amount, by weight, of from about 0.25% to about 20%, based on the total weight of the second treatment composition.

20. The agent for treating keratinous fibers according to claim 19, wherein the wherein the at least one hair active agent is chosen from oxidizing agents, reducing agents, neutralizing agents, dyeing agents, and mixtures thereof.

21. The agent for treating keratinous fibers according to claim 19, further comprising a conditioning composition containing at least one conditioning agent chosen from cationic conditioning agents, silicone compounds, and mixtures thereof.

22. The agent for treating keratinous fibers according to claim 21, wherein the cationic conditioning agents are chosen from monoalkyl quaternary amines, dialkyl quaternary amines, amidoamines in cationic form, polyquarternium compounds, and mixtures thereof.

23. The agent for treating keratinous fibers according to claim 21, wherein the silicone compounds are chosen from polydimethylsiloxanes, polydimethyl siloxanes having hydroxyl end groups, amino functional silicones, and mixtures thereof.

24. The agent for treating keratinous fibers according to claim 19, wherein the first treatment composition is a bleach composition or a coloring composition or a lightening composition or a highlighting composition or a relaxer composition or a straightening composition or a perm composition or a waving composition or neutralizing composition.

25. A method of treating keratinous fibers, comprising:
(i) applying the first treatment composition of claim 19 onto the fibers;
(ii) rinsing the fibers with water; and
(iii) applying the second treatment composition onto the fibers;
wherein the keratinous fibers include hair.

26. The method according to claim 25, further comprising applying onto the fibers, the conditioning composition of claim 20 before rinsing the fibers.

27. A method for treating keratinous fibers comprising applying to the fibers at least two of each of the following components, together, separately, or in any combination thereof, in one or more treatment compositions:
(a) at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and mixtures thereof;
(b) at least one amine chosen from alkyl amines, alkanolamines, alkylene amines, and mixtures thereof;
(c) at least one polycarbodiimide compound; and
(d) at least one carboxysilicone polymer.

28. The method according to claim 27, wherein the at least one amine is chosen from monoethanolamine (MEA), diethanolamine (DEA), triethanolamine (TEA), monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, 3-isopropoxypropylamine, 3-methoxypropylamine (3-MPA), tris(hydroxymethyl)aminomethane, 3-ethoxypropylamine, 3-(2-ethylhexyloxy)-propylamine, 2-(2-aminoethoxy)ethanol (2-2AEE), 3-butoxypropylamine (3-BPA), ethylamines, ethyleneamine, and mixtures thereof.

29. The method according to claim 27, wherein the at least one polycarbodiimide compound has the following formula: 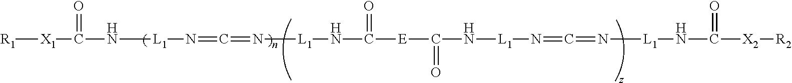
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

30. The method according to claim 27, wherein the at least one carboxysilicone polymer is a compound having at least one carboxylate group and is chosen from:
(A) a compound having the following formula: 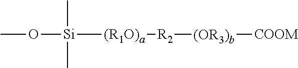
wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or
(B) a compound having a group comprising at least one pyrrolidone carboxylic acid unit having the following formula: 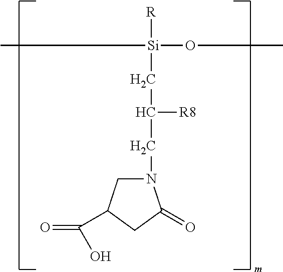
wherein R is selected from methyl or phenyl; R8 is hydrogen or methyl, m is an integer from 1 to 1000;
(C) a compound having a group comprising at least one polyvinyl acid/ester unit resulting from the polymerization of Divinyl-PDMS, Crotonic Acid, Vinylacetate, and Vinyl Isoalkylester;
and combinations thereof.

31. The method according to claim 27, comprising applying to the fibers a first treatment composition containing the at least one carboxylic acid, the at least one amine, and the at least one polycarbodiimide compound.

32. The method according to claim 31, comprising the steps of:
(i) applying to the fibers a first treatment composition containing the at least one carboxylic acid, the at least one amine, and the at least one polycarbodiimide compound;
(ii) heating the fibers at a temperature above room temperature;
(iii) applying to the fibers a second treatment composition containing the at least one polycarbodiimide compound and the at least one carboxysilicone polymer.

33. The method according to claim 27, comprising the steps of:
(i) applying to the fibers a first treatment composition containing the at least one carboxylic acid, the at least one amine, and the at least one polycarbodiimide compound and leaving the first treatment composition on the fibers at room temperature for at least 30 minutes;
(ii) applying to the fibers a second treatment composition containing the at least one polycarbodiimide compound;
iii) applying to the fibers a third treatment composition containing the at least one carboxysilicone polymer and heating the fibers at a temperature above room temperature.

34. The method according to claim 27, comprising:
applying to the fibers a first treatment composition containing the at least one carboxylic acid, the at least one amine, and at least one hair active agent chosen from oxidizing agents, reducing agents, neutralizing agents, dyeing agents, and mixtures thereof; and the method further comprises the steps of:
rinsing the fibers after applying the first composition onto the fibers; and
applying a second composition containing the at least one polycarbodiimide compound and the at least one carboxysilicone polymer.

35. The method according to claim 34, further comprising a step of applying to the fibers a conditioning composition containing at least one conditioning agent chosen from cationic conditioning agents, silicone compounds, and mixtures thereof before the step of rinsing.

36. The method according to claim 27, wherein the method imparts to the keratinous fibers, one or more of:
Improved conditioning;
protection from damage or reduced damage caused by extrinsic and/or intrinsic factors;
repair when the keratinous fibers are in a damaged condition;
minimized or no breakage;
increased or improved mechanical strength;
hydrophobicity or increased hydrophobicity; or
increased hold to the shape or configuration of the fibers;
wherein the keratinous fibers include hair.

37. A kit for treating hair comprising one or more separately contained compositions, wherein each of the one or more compositions include at least one of the following components:
(a) at least one carboxylic acid chosen from maleic acid, oxalic acid, malonic acid, malic acid, glutaric acid, citraconic acid, citric acid, glycolic acid, succinic acid, adipic acid, tartaric acid, fumaric acid, sebacic acid, benzoic acid, glyoxylic acid monohydrate, isocitric acid, aconitic acid, propane-1,2,3-tricarboxylic acid, trimesic acid, a salt thereof, and mixtures thereof;
(b) at least one amine chosen from monoethanolamine, diethanolamine, triethanolamine, 3-isopropoxypropylamine, 3-methoxypropylamine, tris(hydroxymethyl)aminomethane, 3-ethoxypropylamine, 3-(2-ethylhexyloxy)-propylamine, 2-(2-aminoethoxy)ethanol, 3-butoxypropylamine, and mixtures thereof;
(c) at least one polycarbodiimide compound having the following formula: 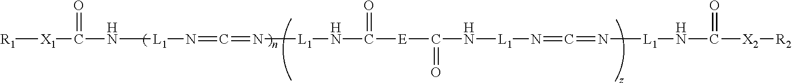
wherein X₁ and X₂, each independently, represents O, S or NH; R, and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group, wherein a plurality of L₁ groups may be identical to or different from one another; E is a radical selected from:
O—R₃—O; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms; and
(d) at least one carboxysilicone polymer, based on the total weight of the composition and is a compound having at least one carboxylate group and is chosen from:
(A) a compound having the following formula: 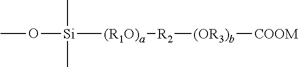
wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or
(B) a compound having a group comprising at least one pyrrolidone carboxylic acid unit having the following formula: 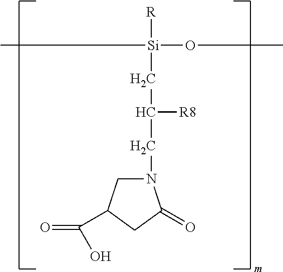
wherein R is selected from methyl or phenyl; R8 is hydrogen or methyl, m is an integer from 1 to 1000;
(C) a compound having a group comprising at least one polyvinyl acid/ester unit resulting from the polymerization of Divinyl-PDMS, Crotonic Acid, Vinylacetate, and Vinyl Isoalkylester;
and combinations thereof.
